(19) United States
(12) Patent Application Publication
(10) Pub. No.: US 2026/0298917 A1
(43) Pub. Date: Oct. 1, 2026

(54) NANOPARTICLE ENHANCED METHODS AND MATERIALS FOR DETECTING MISFOLDED POLYPEPTIDES

(71) Applicant: Regents of the University of Minnesota, Minneapolis, MN (US)

(72) Inventors: Sang-Hyun Oh, Minneapolis, MN (US); Peter A. Larsen, Minneapolis, MN (US); Peter C. Christenson, Minneapolis, MN (US); Manci Li, Minneapolis, MN (US); Gage Rowden, Minneapolis, MN (US)

(21) Appl. No.: 19/475,911

(22) PCT Filed: Apr. 18, 2024
(86) PCT No.: PCT/US2024/025182

§ 371 Date: Oct. 16, 2025

Related U.S. Application Data
(60) Provisional application No. 63/460,179, filed on Apr. 18, 2023.

(51) Int. Cl. G01N 33/543

(52) U.S. Cl. CPC G01N 33/54346

(57) ABSTRACT

This document relates to methods and materials for detecting the presence or absence of misfolded polypeptides in a sample. For example, a sample (e.g., a biological sample or an environmental sample) can be exposed to nanoparticles (e.g., nanoparticles having a size of no more than 2 μm (e.g., no more than 1 μm) such as silica nanoparticles (siNPs) having a size of no more than 2 μm (e.g., siNPs having a size of no more than 1 μm)) during a seeded amplification assay to accelerate the aggregation of misfolded polypeptides present in the sample into fibrils and/or polypeptide aggregates (e.g., globular polypeptide aggregates). In some cases, methods and materials provided herein can be used to determine if a mammal (e.g., a human) has a proteinopathy based, at least in part, in the presence or absence of misfolded polypeptides in a sample obtained from the mammal.

### CROSS-REFERENCE TO RELATED APPLICATIONS

This application claims the benefit of U.S. Patent Application Ser. No. 63/460,179, filed on Apr. 18, 2023. The disclosure of the prior application is considered part of, and is incorporated by reference in, the disclosure of this application.

### TECHNICAL FIELD

This document relates to methods and materials for detecting the presence or absence of misfolded polypeptides in a sample. For example, a sample (e.g., a biological sample or an environmental sample) can be exposed to nanoparticles (e.g., nanoparticles having a size of no more than 2 μm such as silica nanoparticles (siNPs) having a size of no more than 2 μm)) during a seeded amplification assay to accelerate the aggregation of misfolded polypeptides present in the sample into fibrils and/or polypeptide aggregates (e.g., globular polypeptide aggregates). For example, a sample (e.g., a biological sample or an environmental sample) can be exposed to nanoparticles (e.g., nanoparticles having a size of no more than 1 μm such as siNPs having a size of no more than 1 μm) during a seeded amplification assay to accelerate the aggregation of misfolded polypeptides present in the sample into fibrils and/or polypeptide aggregates (e.g., globular polypeptide aggregates). In some cases, methods and materials provided herein can be used to determine if a mammal (e.g., a human) has a proteinopathy based, at least in part, in the presence or absence of misfolded polypeptides in a sample obtained from the mammal.

### BACKGROUND

Pathological amyloids formed by misfolded polypeptides are found in many neurodegenerative diseases of both humans and animals, including Alzheimer's Disease (misfolded Aβ and tau), Parkinson's disease (misfolded α-synuclein), and prion diseases (misfolded cellular prion proteins, PrP^{C}) (Prusiner et al., Proc. Natl. Acad. Sci. USA, 95 (23), 13363-13383 (1998); Soto et al., Arch. Neurol., 65 (2), 184-189 (2008); Marsh et al., J. Virol., 79 (21), 13794-13796 (2005); and Kim et al., Sci. Rep., 6:19548 (2016)). Amyloid formation observed across pathogenic protein isoforms originates via the misfolding of functional polypeptides into insoluble and degradation-resistant amyloid fibrils. A notable feature across the spectrum of neurodegenerative disease is that the production and deposition of affiliated misfolded polypeptides can begin years before the onset of clinical symptoms.

Real-time quaking-induced conversion (RT-QuIC) has emerged as one of the most sensitive and promising assays for the early diagnosis of various misfolded polypeptide-related neurodegenerative diseases (Iranzo et al., Lancet Neurol., 20 (3), 203-212 (2021); Dong et al., Pathogens, 10 (3): 305 (2021); Coysh et al., Front. Aging Neurosci., 14, 872629 (2022); Telling et al., Proc. Natl. Acad. Sci. USA, 116 (46), 22894-22896 (2019); and Haley et al., Pathogens, 6 (3): 35 (2017)). While RT-QuIC has been successfully applied to detect several misfolded polypeptides associated with a variety of neurodegenerative diseases (e.g., TDP-43, Tau, Alpha-synuclein, infectious prions, etc.), the complexity of biological samples still poses challenges for clinical diagnostic applications. For example, the assay often takes up to 48 hours to complete a standard test. Additionally, natural inhibitors present within biological samples can interfere with reaction kinetics, producing false-negative results (Davenport et al., J. Clin. Microbiol., 56 (9): e00947-18 (2018); Coysh et al., Front. Aging Neurosci., 14, 872629 (2022); Li et al., Sci. Rep., 11 (1): 16759 (2021); and Orrù et al., “Real-Time Quaking-Induced Conversion (QuIC) Assays for the Detection and Diagnosis of Human Prion Diseases.” pages 621-635 in Prions and Diseases, Zou et al., (Eds); Springer International Publishing: Cham, 2023).

### SUMMARY

This document provides methods and materials for detecting the presence or absence of misfolded polypeptides, polypeptide fibrils, and/or polypeptide aggregates (e.g., globular polypeptide aggregates) in a sample. Misfolded polypeptides can accumulate to form aggregates (e.g., aggregates of two or more misfolded polypeptides) such as fibrils (e.g., amyloid fibrils), polypeptide aggregates (e.g., globular polypeptide aggregates), and/or oligomers. For example, a sample (e.g., a biological sample or an environmental sample) can be exposed to nanoparticles (e.g., nanoparticles having a size of no more than 2 μm such as siNPs having a size of no more than 2 μm) during a seeded amplification assay to accelerate the aggregation of misfolded polypeptides present in the sample into fibrils and/or polypeptide aggregates. For example, a sample (e.g., a biological sample or an environmental sample) can be exposed to nanoparticles (e.g., nanoparticles having a size of no more than 1 μm such as siNPs having a size of no more than 1 μm) during a seeded amplification assay to accelerate the aggregation of misfolded polypeptides present in the sample into fibrils and/or polypeptide aggregates. Seeded amplification assays that are accelerated by the presence of nanoparticles can be referred to as nanoparticle enhanced seeded amplification assays such as nanoparticle enhanced RT-QuIC (Nano-QuIC).

In some cases, a sample (e.g., a biological sample or an environmental sample) can be exposed to nanoparticles (e.g., nanoparticles having a size of no more than 2 μm (e.g., no more than 1 μm) such as siNPs having a size of no more than 2 μm (e.g., no more than 1 μm)) during a seeded amplification assay. For example, a seeded amplification assay can include (a) contacting a sample (e.g., a sample suspected of containing misfolded polypeptides) with a polypeptide substrate (e.g., a recombinant PrP substrate such as a recombinant hamster PrP (rPrP)) to form a mixture, and (b) agitating the mixture for a period of time. In some cases, methods and materials provided herein can be used to determine if a mammal (e.g., a human) has a proteinopathy based, at least in part, in the presence or absence of misfolded polypeptides in a sample obtained from the mammal. For example, a sample obtained from a mammal can be exposed to nanoparticles (e.g., nanoparticles having a size of no more than 2 μm (e.g., no more than 1 μm) such as siNPs having a size of no more than 2 μm (e.g., no more than 1 μm)) during a seeded amplification assay to accelerate the aggregation of misfolded polypeptides present in the sample into fibrils and/or polypeptide aggregates, the presence or absence of fibrils and/or polypeptide aggregates can be detected, and the mammal can be classified as having a proteinopathy if the presence of fibrils and/or polypeptide aggregates is detected.

As demonstrated herein, adding nanoparticles (e.g., siNPs) having a size of no more than 2 μm (e.g., no more than 1 μm) to a seeded amplification assay (e.g., a RT-QuIC reaction) can accelerate the amplification (e.g., the aggregation of misfolded polypeptides). For example, adding nanoparticles (e.g., siNPs) having a size of no more than 2 μm (e.g., no more than 1 μm) to a RT-QuIC reaction can increase the rate of amyloid formation during an RT-QuIC reaction. In some cases, adding nanoparticles (e.g., siNPs) having a size of no more than 2 μm (e.g., no more than 1 μm) to a RT-QuIC reaction also can increase the sensitivity of the RT-QuIC reaction.

Having the ability to detect the presence of misfolded polypeptides in a sample as described herein (e.g., by exposing the sample to nanoparticles (e.g., siNPs) having a size of no more than 2 μm (e.g., no more than 1 μm) during a seeded amplification assay) provides an accelerated method to quickly and effectively detect the presence of misfolded polypeptides. For example, the methods described herein provide a protein-based diagnostic method that is relatively easy to use, requires inexpensive reagents and equipment, is highly sensitive and specific to the targeted protein, can be performed in diverse settings (e.g., in field settings, small laboratories, etc.), and can be performed in about 4 hours or less (e.g., in about 4 hours, in a about 3 hours, in about 2 hours, or less). In some cases, the methods and materials described herein can be used to quickly and easily identify a mammal as having a proteinopathy.

In general, one aspect of this document features methods for detecting the presence or absence of misfolded polypeptides in a sample where the methods can include, or consist essentially of, (a) exposing a sample to nanoparticles having a size of no more than 2 μm (e.g., no more than 1 μm) during a seeded amplification assay, wherein misfolded polypeptides, when present within said sample, form fibrils; (b) detecting said fibrils if formed in step (a); (c) identifying said sample as having said presence of said misfolded polypeptides if said fibrils are detected; and (d) identifying said sample as lacking said misfolded polypeptides if said fibrils are not detected. The sample can be a biological sample. The biological sample can be obtained from a living mammal. The living mammal can be a human, a monkey, a camel, a horse, a mink, a cat, a canine, a cow, a sheep, a mouse, a rat, a hamster, a brocket, a chital, an elk, a fallow deer, a marsh deer, a mule deer, a muntjac, a moose, a pampas deer, a red deer, a reindeer, a roe deer, a sambar deer, a sika, a white-tailed deer, an antelope, or a goat. The biological sample can be lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, semen, spleen tissue, or eye tissue. The biological sample can be obtained from a mammal post-mortem. The biological sample can be beef, mutton, lamb, or venison. The sample can be an environmental sample. The environmental sample can be soil, water, dust, or plants. The environmental sample can be obtained using a swab or a filter. The environmental sample can be obtained from a location selected from group consisting of a natural habitat, a waterway, a farm, a food processing facility, a water-treatment facility, and a medical facility. The environmental sample can be obtained from said food processing facility, and said food processing facility processes food intended for mammalian consumption. The environmental sample can be obtained from said medical facility. The method can include, prior to said step (a), isolating polypeptides from said sample. The seeded amplification assay can include shaking said sample, sonicating said sample, exposing said sample to acoustic waves, or light illumination of said sample. The misfolded polypeptide can be prion protein (PrP) polypeptides, tau polypeptides, amyloid β polypeptides, α-synuclein polypeptides, TDP-43 polypeptides, islet amyloid polypeptides (IAPPs), superoxide dismutase 1 (SOD1) polypeptides, huntingtin polypeptides, fused in sarcoma (FUS) polypeptides, or translocated in liposarcoma (TLS) polypeptides. The misfolded polypeptide can be associated with a proteinopathy. The proteinopathy can be chronic wasting disease (CWD), Creutzfeldt-Jakob Disease, transmissible mink encephalopathy, scrapie, feline spongiform encephalopathy, ungulate spongiform encephalopathy, bovine-spongiform encephalopathy, camelid spongiform encephalopathy, pituitary pars intermedia dysfunction (PPID), Alzheimer's Disease (AD), Parkinson's Disease (PD), Pick's disease, Lewy body dementia (LBD), amyotrophic lateral sclerosis (ALS), multiple systems atrophies, progressive supranuclear palsies, corticobasal degenerations, or chronic traumatic encephalopathies. The seeded amplification assay can be completed in less than about 500 hours. The seeded amplification assay can be completed in less than about 96 hours. The nanoparticles can be silica nanoparticles, gold nanoparticles, silver nanoparticles, platinum nanoparticles, iron nanoparticles, copper nanoparticles, dielectric nanoparticles with metallic shells, titanium dioxide nanoparticles, tin dioxide nanoparticles, cerium oxide nanoparticles, zirconium dioxide nanoparticles, iron oxide nanoparticles, cadmium telluride quantum dots, or lead sulfide quantum dots.

In another aspect, this document features methods for detecting the presence or absence of misfolded polypeptides in a sample where the methods can include, or consist essentially of, (a) exposing said sample to nanoparticles having a size of no more than 2 μm (e.g., no more than 1 μm) during a seeded amplification assay, wherein misfolded polypeptides, when present within said sample, form globular polypeptide aggregates; (b) detecting said globular polypeptide aggregates if formed in step (a); (c) identifying said sample as having said presence of said misfolded polypeptides if said globular polypeptide aggregates are detected; and (d) identifying said sample as lacking said misfolded polypeptides if said globular polypeptide aggregates are not detected. The sample can be a biological sample. The biological sample can be obtained from a living mammal. The living mammal can be a human, a monkey, a camel, a horse, a mink, a cat, a canine, a cow, a sheep, a mouse, a rat, a hamster, a brocket, a chital, an elk, a fallow deer, a marsh deer, a mule deer, a muntjac, a moose, a pampas deer, a red deer, a reindeer, a roe deer, a sambar deer, a sika, a white-tailed deer, an antelope, or a goat. The biological sample can be lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, semen, spleen tissue, or eye tissue. The biological sample can be obtained from a mammal post-mortem. The biological sample can be beef, mutton, lamb, or venison. The sample can be an environmental sample. The environmental sample can be soil, water, dust, or plants. The environmental sample can be obtained using a swab or a filter. The environmental sample can be obtained from a location selected from group consisting of a natural habitat, a waterway, a farm, a food processing facility, a water-treatment facility, and a medical facility. The environmental sample can be obtained from said food processing facility, and said food processing facility processes food intended for mammalian consumption. The environmental sample can be obtained from said medical facility. The method can include, prior to said step (a), isolating polypeptides from said sample. The seeded amplification assay can include shaking said sample, sonicating said sample, exposing said sample to acoustic waves, or light illumination of said sample. The misfolded polypeptide can be PrP polypeptides, tau polypeptides, amyloid β polypeptides, α-synuclein polypeptides, TDP-43 polypeptides, IAPPs, SOD1 polypeptides, huntingtin polypeptides, FUS polypeptides, or TLS polypeptides. The misfolded polypeptide can be associated with a proteinopathy. The proteinopathy can be CWD, Creutzfeldt-Jakob Disease, transmissible mink encephalopathy, scrapie, feline spongiform encephalopathy, ungulate spongiform encephalopathy, bovine-spongiform encephalopathy, camelid spongiform encephalopathy, PPID, AD, PD, Pick's disease, LBD, ALS, multiple systems atrophies, progressive supranuclear palsies, corticobasal degenerations, or chronic traumatic encephalopathies. The seeded amplification assay can be completed in less than about 500 hours. The seeded amplification assay can be completed in less than about 96 hours. The nanoparticles can be silica nanoparticles, gold nanoparticles, silver nanoparticles, platinum nanoparticles, iron nanoparticles, copper nanoparticles, dielectric nanoparticles with metallic shells, titanium dioxide nanoparticles, tin dioxide nanoparticles, cerium oxide nanoparticles, zirconium dioxide nanoparticles, iron oxide nanoparticles, cadmium telluride quantum dots, or lead sulfide quantum dots.

In another aspect, this document features compositions including nanoparticles having a size of no more than 2 μm (e.g., no more than 1 μm) for use in accelerating a seeded amplification assay. The seeded amplification assay can include shaking said sample, sonicating said sample, exposing said sample to acoustic waves, or light illumination of said sample. The nanoparticles can be silica nanoparticles, gold nanoparticles, silver nanoparticles, platinum nanoparticles, iron nanoparticles, copper nanoparticles, dielectric nanoparticles with metallic shells, titanium dioxide nanoparticles, tin dioxide nanoparticles, cerium oxide nanoparticles, zirconium dioxide nanoparticles, iron oxide nanoparticles, cadmium telluride quantum dots, or lead sulfide quantum dots. The seeded amplification assay can be completed in less than about 500 hours. The seeded amplification assay can be completed in less than about 96 hours.

In another aspect, this document features methods for detecting the presence or absence of misfolded polypeptides in a sample, wherein the methods can include, or consist essentially of, (a) exposing said sample to a nanoporous material having a pore size of from 2 nm to about 200 nm (e.g., from 2 nm to about 100 nm) during a seeded amplification assay, wherein misfolded polypeptides, when present within said sample, form fibrils; (b) detecting said fibrils if formed in step (a); (c) identifying said sample as having said presence of said misfolded polypeptides if said fibrils are detected; and (d) identifying said sample as lacking said misfolded polypeptides if said fibrils are not detected. The sample can be a biological sample. The biological sample can be obtained from a living mammal. The living mammal can be a human, a monkey, a camel, a horse, a mink, a cat, a canine, a cow, a sheep, a mouse, a rat, a hamster, a brocket, a chital, an elk, a fallow deer, a marsh deer, a mule deer, a muntjac, a moose, a pampas deer, a red deer, a reindeer, a roe deer, a sambar deer, a sika, a white-tailed deer, an antelope, or a goat. The biological sample can be lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, semen, spleen tissue, or eye tissue. The biological sample can be obtained from a mammal post-mortem. The biological sample can be beef, mutton, lamb, or venison. The sample can be an environmental sample. The environmental sample can be soil, water, dust, or plants. The environmental sample can be obtained using a swab or a filter. The environmental sample can be obtained from a location selected from group consisting of a natural habitat, a waterway, a farm, a food processing facility, a water-treatment facility, and a medical facility. The environmental sample can be obtained from said food processing facility, and wherein said food processing facility processes food intended for mammalian consumption. The method of claim 56, wherein said environmental sample is obtained from said medical facility. The method can include, prior to said step (a), isolating polypeptides from said sample. The seeded amplification assay can include shaking said sample, sonicating said sample, exposing said sample to acoustic waves, or light illumination of said sample. The misfolded polypeptide can be PrP polypeptides, tau polypeptides, amyloid β polypeptides, α-synuclein polypeptides, TDP-43 polypeptides, IAPPs, SOD1 polypeptides, huntingtin polypeptides, FUS polypeptides, or TLS polypeptides. The misfolded polypeptide can be associated with a proteinopathy. The proteinopathy can be CWD, Creutzfeldt-Jakob Disease, transmissible mink encephalopathy, scrapie, feline spongiform encephalopathy, ungulate spongiform encephalopathy, bovine-spongiform encephalopathy, camelid spongiform encephalopathy, PPID, AD, PD, Pick's disease, LBD, ALS, multiple systems atrophies, progressive supranuclear palsies, corticobasal degenerations, or chronic traumatic encephalopathies. The seeded amplification assay can be completed in less than about 500 hours. The seeded amplification assay can be completed in less than about 96 hours. The nanoporous material can be mesoporous silica, mesoporous polytetrafluoroethylene, zeolite particles, or metal-organic frameworks (MOFs).

In another aspect, this document features methods for detecting the presence or absence of misfolded polypeptides in a sample where the methods can include, or consist essentially of, (a) exposing said sample to a nanoporous material having a pore size of from 2 nm to about 200 nm (e.g., from 2 nm to about 100 nm) during a seeded amplification assay, wherein misfolded polypeptides, when present within said sample, form globular polypeptide aggregates; (b) detecting said globular polypeptide aggregates if formed in step (a); (c) identifying said sample as having said presence of said misfolded polypeptides if said globular polypeptide aggregates are detected; and (d) identifying said sample as lacking said misfolded polypeptides if said globular polypeptide aggregates are not detected. The sample can be a biological sample. The biological sample can be obtained from a living mammal. The living mammal can be a human, a monkey, a camel, a horse, a mink, a cat, a canine, a cow, a sheep, a mouse, a rat, a hamster, a brocket, a chital, an elk, a fallow deer, a marsh deer, a mule deer, a muntjac, a moose, a pampas deer, a red deer, a reindeer, a roe deer, a sambar deer, a sika, a white-tailed deer, an antelope, or a goat. The biological sample can be lymph tissue, muscle tissue, tonsil tissue, skin tissue, brain tissue, brain-stem tissue, blood, cerebrospinal fluid, urine, feces, saliva, mucus, liver tissue, heart tissue, intestinal tissue, semen, spleen tissue, or eye tissue. The biological sample can be obtained from a mammal post-mortem. The biological sample can be beef, mutton, lamb, or venison. The sample can be an environmental sample. The environmental sample can be soil, water, dust, or plants. The environmental sample can be obtained using a swab or a filter. The environmental sample can be obtained from a location selected from group consisting of a natural habitat, a waterway, a farm, a food processing facility, a water-treatment facility, and a medical facility. The environmental sample can be obtained from said food processing facility, and said food processing facility processes food intended for mammalian consumption. The environmental sample can be obtained from said medical facility. The method can include, prior to said step (a), isolating polypeptides from said sample. The seeded amplification assay can include shaking said sample, sonicating said sample, exposing said sample to acoustic waves, or light illumination of said sample. The misfolded polypeptide can be PrP polypeptides, tau polypeptides, amyloid β polypeptides, α-synuclein polypeptides, TDP-43 polypeptides, IAPPs, SOD1 polypeptides, huntingtin polypeptides, FUS polypeptides, or TLS polypeptides. The misfolded polypeptide can be associated with a proteinopathy. The proteinopathy can be CWD, Creutzfeldt-Jakob Disease, transmissible mink encephalopathy, scrapie, feline spongiform encephalopathy, ungulate spongiform encephalopathy, bovine-spongiform encephalopathy, camelid spongiform encephalopathy, PPID, AD, PD, Pick's disease, LBD, ALS, multiple systems atrophies, progressive supranuclear palsies, corticobasal degenerations, aornd chronic traumatic encephalopathies. The seeded amplification assay can be completed in less than about 500 hours. The seeded amplification assay can be completed in less than about 96 hours. The nanoporous material can be mesoporous silica, mesoporous polytetrafluoroethylene, zeolite particles, or MOFs.

In another aspect, this document features compositions including a nanoporous material having a pore size of from 2 nm to about 200 nm (e.g., from 2 nm to about 100 nm) for use in accelerating a seeded amplification assay. The seeded amplification assay can include shaking said sample, sonicating said sample, exposing said sample to acoustic waves, or light illumination of said sample. The nanoporous material can be mesoporous silica, mesoporous polytetrafluoroethylene, zeolite particles, or MOFs. The seeded amplification assay can be completed in less than about 500 hours. The seeded amplification assay can be completed in less than about 96 hours.

In another aspect, this document features methods for detecting the presence or absence of misfolded polypeptides in a sample where the methods can include, or consist essentially of, (a) exposing said sample to nanoparticles having a size of no more than 2 μm (e.g., no more than 1 μm) during a seeded amplification assay comprising exposing said sample to acoustic waves, wherein misfolded polypeptides, when present within said sample, form fibrils; (b) detecting said fibrils if formed in step (a); (c) identifying said sample as having said presence of said misfolded polypeptides if said fibrils are detected; and (d) identifying said sample as lacking said misfolded polypeptides if said fibrils are not detected.

In another aspect, this document features methods for detecting the presence or absence of misfolded polypeptides in a sample where the methods can include, or consist essentially of, (a) exposing said sample to nanoparticles having a size of no more than 2 μm (e.g., no more than 1 μm) during a seeded amplification assay comprising exposing said sample to acoustic waves, wherein misfolded polypeptides, when present within said sample, form globular polypeptide aggregates; (b) detecting said globular polypeptide aggregates if formed in step (a); (c) identifying said sample as having said presence of said misfolded polypeptides if said globular polypeptide aggregates are detected; and (d) identifying said sample as lacking said misfolded polypeptides if said globular polypeptide aggregates are not detected.

In another aspect, this document features methods for detecting the presence or absence of misfolded polypeptides in a sample where the methods can include, or consist essentially of, (a) exposing said sample to metallic nanoparticles having a size of no more than 2 μm (e.g., no more than 1 μm) during a seeded amplification assay comprising light illumination of said sample, wherein misfolded polypeptides, when present within said sample, form fibrils; (b) detecting said fibrils if formed in step (a); (c) identifying said sample as having said presence of said misfolded polypeptides if said fibrils are detected; and (d) identifying said sample as lacking said misfolded polypeptides if said fibrils are not detected.

In another aspect, this document features methods for detecting the presence or absence of misfolded polypeptides in a sample where the methods can include, or consist essentially of, (a) exposing said sample to metallic nanoparticles having a size of no more than 2 μm (e.g., no more than 1 μm) during a seeded amplification assay comprising light illumination of said sample, wherein misfolded polypeptides, when present within said sample, form globular polypeptide aggregates; (b) detecting said globular polypeptide aggregates if formed in step (a); (c) identifying said sample as having said presence of said misfolded polypeptides if said globular polypeptide aggregates are detected; and (d) identifying said sample as lacking said misfolded polypeptides if said globular polypeptide aggregates are not detected.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1C: FIG. 1A) Summary of fibril growth showing nucleation, growth, and stationary phases. Sigmoidal curve represents misfolded protein (MP) detection, flat line represents no MP detection. FIG. 1B) Results from Nano-QuIC and traditional RT-QuIC. Nano-QuIC conditions: 48° C., 2.5 mg/mL 50 nm silica NPs. FIG. 1C) Schematic of the Nano-QuIC seeded-amplification mechanism. In Nano-QuIC, silica NPs promote primary nucleation at a higher rate than the bulk solution methods such as RT-QuIC.

FIGS. 2A-2D: FIG. 2A) Relative rate of amyloid formation (RAF) of various diameter and concentration of silica NP solutions amplified at 42° C. seeded with CWD positive or negative tissue homogenates. FIG. 2B) Relative RAF of solutions seeded with CWD positive or negative tissue with various concentrations of 50 nm silica NPs amplified at different temperatures. FIG. 2C) The lag time of the nucleation phase vs concentration of 50 nm silica NPs at 48° C. FIG. 2D) The time constant of fibril growth (t) vs concentration of 50 nm silica NPs at 48° C. Unless noted otherwise error bars show standard deviation.

FIG. 3: Relative RAF (ratio of the RAF of reactions with 15 nm AuNPs compared to those without). Assay ran at 42° C. Error bars show standard deviation.

FIGS. 4A-4B: FIG. 4A) Raw RAF for a ten-fold dilution series of CWD positive tissue amplified at 42° C. in RT-QuIC vs. Nano-QuIC (50 nm siNPs, 2.5 mg/mL). Arrow highlights the lack of signal (false negative) from RT-QuIC, likely due to inhibitors. FIG. 4B) Kinetic curves for 10⁻¹ CWD positive tissue dilution amplified at 42° C. in RT-QUIC vs Nano-QuIC (50 nm siNPs, 2.5 mg/mL). Unless noted otherwise error bars show standard deviation.

FIGS. 5A-5B: FIG. 5A) Relative RAF of Nano-QuIC vs various temperature and concentrations of 20 nm silica NPs. FIG. 5B) Relative RAF of Nano-QuIC vs various temperature and concentrations of 100 nm silica NPs.

FIGS. 6: The kinetic curves for Nano-QuIC (50 nm, 2.5 mg/ml, 48° C.) for positive and negative CWD Samples.

FIGS. 7A-7C: Illustration demonstrating that the addition of nanoparticles (NPs) with diameters less than one micron accelerates the detection of misfolded polypeptides by seeded amplification assays (SAAs). FIG. 7A) Graphic explanation of SAAs. SAAs are highly sensitive assays used to detect misfolded protein seeds through amplifying protein misfolding. SAAs involves multiple cycles of amplifying protein misfolding. In each cycle, when misfolded protein seeds are present, the assay provides protein substrates that support the growth of the misfolded protein fibrils (amyloids) initiated by the seeds. Next, mechanical forces (e.g., shaking or sonication) are provided to fragment the misfolded protein fibrils to create more seeds for the next cycle of fibril amplification. FIG. 7B) The addition of NPs reduced the SAA cycles needed to achieve the detection of misfolded polypeptides. ThT, Thioflavin T, a fluorophore used to detect misfolded polypeptides; NPs, nanoparticles with diameters less than 1 micron; Threshold, the fluorescent level used to determine whether misfolded polypeptides are detected. FIG. 7C) The addition of NPs reduced the rate of amyloid formation needed to achieve the detection of misfolded polypeptides. Rate of amyloid formation=1/time to threshold, a term used to describe how fast amyloids formed in SAA.

FIGS. 8A-8B: Nano-QuIC applied to human samples. FIG. 8A) Comparison of Nano-QuIC and conventional RT-QuIC for detecting artificially misfolded α-synuclein (the protein associated with Parkinson's disease) in human plasma with final spike concentrations ranging from 9 μg/mL down to 90 μg/mL. Nano-QuIC overcomes inhibitors to allow a 100-fold improvement in sensitivity. FIG. 8B) Nano-QuIC data of two human plasma samples made up of patients diagnosed with Parkinson's disease (solid line) versus a healthy control (dotted line). Nano-QuIC detected more seeding activity in the Parkinson's samples than the healthy control.

### DETAILED DESCRIPTION

This document provides methods and materials for detecting the presence or absence of misfolded polypeptides, polypeptide fibrils, and/or polypeptide aggregates (e.g., globular polypeptide aggregates) in a sample. In some cases, a sample (e.g., a biological sample or an environmental sample) can be exposed to nanoparticles (e.g., nanoparticles having a size of no more than 2 μm such as siNPs having a size of no more than 2 μm) during a seeded amplification assay to accelerate the aggregation of misfolded polypeptides present in the sample into fibrils and/or polypeptide aggregates, and the presence or absence of fibrils and/or polypeptide aggregates can be detected. For example, a sample suspected of containing misfolded polypeptides can be exposed to nanoparticles (e.g., nanoparticles having a size of no more than 2 μm such as siNPs having a size of no more than 2 μm) during a seeded amplification assay such that misfolded polypeptides, when present, can aggregate to form fibrils (e.g., aggregates of two or more misfolded polypeptides), and the presence or absence of fibrils can be detected. In some cases, a sample (e.g., a biological sample or an environmental sample) can be exposed to nanoparticles (e.g., nanoparticles having a size of no more than 1 μm such as siNPs having a size of no more than 1 μm) during a seeded amplification assay to accelerate the aggregation of misfolded polypeptides present in the sample into fibrils and/or polypeptide aggregates, and the presence or absence of fibrils and/or polypeptide aggregates can be detected. For example, a sample suspected of containing misfolded polypeptides can be exposed to nanoparticles (e.g., nanoparticles having a size of no more than 1 μm such as siNPs having a size of no more than 1 μm) during a seeded amplification assay such that misfolded polypeptides, when present, can aggregate to form fibrils (e.g., aggregates of two or more misfolded polypeptides), and the presence or absence of fibrils can be detected.

A sample (e.g., a biological sample or an environmental sample) can be exposed to nanoparticles (e.g., nanoparticles having a size of no more than 2 μm (e.g., no more than 1 μm) such as siNPs having a size of no more than 2 μm (e.g., no more than 1 μm)) in any type of seeded amplification assay. In some cases, a seeded amplification assay can include (a) contacting a sample (e.g., a sample suspected of containing misfolded polypeptides) with a polypeptide substrate (e.g., a recombinant PrP substrate such as a recombinant hamster PrP (rPrP)) to form a mixture, and (b) agitating the mixture for a period of time.

A seeded amplification assay can include contacting a sample (e.g., a sample suspected of containing misfolded polypeptides) with any appropriate polypeptide substrate. In some cases, a polypeptide substrate can include one or more recombinant polypeptides. Examples of polypeptide substrates that can be used in a seeded amplification assay include, without limitation, recombinant PrP polypeptides (e.g., rPrP polypeptides) and α-synuclein polypeptides. In some cases, a polypeptide substrate can be in the form of a tissue homogenate (e.g., a tissue homogenate containing one or more polypeptide substrates).

Examples of seeded amplification assays include, without limitation, quaking-induced conversion assays (e.g., RT-QuIC assays, MN-QuIC assays, and photonic-QuIC (Photo-QuIC) assays) and protein-misfolding cyclic amplification (PMCA) assays. In some cases, a sample (e.g., a biological sample or an environmental sample) can be exposed to nanoparticles (e.g., nanoparticles having a size of no more than 2 μm (e.g., no more than 1 μm) such as siNPs having a size of no more than 2 μm (e.g., no more than 1 μm)) in a seeded amplification assay described elsewhere (see, e.g., WO 2002/221683; Haley et al., Pathogens, 6:35 (2017); Dong et al., Pathogens, 10:305 (2021); and Schwabenlander et al., J. Wildl. Dis., 58 (1): 50-62 (2021)). In some cases, a sample (e.g., a biological sample or an environmental sample) can be exposed to nanoparticles (e.g., nanoparticles having a size of no more than 2 μm (e.g., no more than 1 μm) such as siNPs having a size of no more than 2 μm (e.g., no more than 1 μm)) in a seeded amplification assay performed in a microfluidic device.

In some cases, the methods and materials described herein (e.g., the methods for detecting the presence or absence of a misfolded polypeptide) can be used to detect the presence or absence of misfolded polypeptides in less than 500 hours (e.g., in less than 450 hours, in less than 400 hours, in less than 350 hours, in less than 300 hours, in less than 250 hours, in less than 200 hours, in less than 150 hours, in less than 100 hours, or in less than 50 hours). For example, the methods and materials for detecting the presence or absence of a misfolded polypeptide described herein can be used to detect the presence or absence of misfolded polypeptides in from about 2 hours to about 500 hours (e.g., in from about 2 hours to about 400 hours, in from about 2 hours to about 300 hours, in from about 2 hours to about 200 hours, in from about 2 hours to about 100 hours, in from about 2 hours to about 50 hours, in from about 50 hours to about 500 hours, in from about 100 hours to about 500 hours, in from about 150 hours to about 500 hours, in from about 200 hours to about 500 hours, in from about 250 hours to about 500 hours, in from about 300 hours to about 500 hours, in from about 350 hours to about 500 hours, in from about 400 hours to about 500 hours, in from about 450 hours to about 500 hours, in from about 50 hours to about 450 hours, in from about 100 hours to about 400 hours, in from about 150 hours to about 350 hours, in from about 200 hours to about 300 hours, in from about 50 hours to about 150 hours, in from about 100 hours to about 200 hours, in from about 150 hours to about 250 hours, in from about 250 hours to about 350 hours, in from about 300 hours to about 400 hours, or in from about 350 hours to about 450 hours).

In some cases, the methods and materials described herein (e.g., the methods for detecting the presence or absence of a misfolded polypeptide) can be used to detect the presence or absence of misfolded polypeptides in less than 96 hours (e.g., in less than 48 hours, in less than 14 hours, in less than 10 hours, in less than 8 hours, or in less than about 5 hours). For example, the methods and materials for detecting the presence or absence of a misfolded polypeptide described herein can be used to detect the presence or absence of misfolded polypeptides in from about 2 hours to about 96 hours (e.g., from about 2 hours to about 48 hours, from about 2 hours to about 14 hours, from about 2 hours to about 8 hours, from about 2 hours to about 6 hours, from about 2 hours to about 5 hours, from about 2 hours to about 4 hours, from about 4 hours to about 96 hours, from about 6 hours to about 96 hours, from about 12 hours to about 96 hours, from about 3 hours to about 48 hours, from about 5 hours to about 14 hours, from about 3 hours to about 12 hours, from about 4 hours to about 10 hours, from about 5 hours to about 8 hours, from about 3 hours to about 5 hours, from about 4 hours to about 6 hours, from about 5 hours to about 7 hours, from about 6 hours to about 8 hours, from about 7 hours to about 9 hours, from about 8 hours to about 10 hours, or from about 9 hours to about 11 hours).

In some cases, the methods and materials described herein (e.g., the methods for detecting the presence or absence of a misfolded polypeptide) can be used to detect the presence or absence of any misfolded polypeptides, polypeptide fibrils, and/or polypeptide aggregates (e.g., globular polypeptide aggregates). For example, the methods and materials described herein can be used to detect the presence or absence of amyloid plaques. For example, the methods and materials described herein can be used to detect the presence or absence of tau tangles.

In some cases, the methods and materials described herein (e.g., the methods for detecting the presence or absence of a misfolded polypeptide) can be used to determine if a mammal (e.g., a human) has a proteinopathy based, at least in part, in the presence or absence of misfolded polypeptides in a sample obtained from the mammal. For example, a sample obtained from a mammal can be exposed to nanoparticles (e.g., nanoparticles having a size of no more than 2 μm (e.g., no more than 1 μm) such as siNPs having a size of no more than 2 μm (e.g., no more than 1 μm)) during a seeded amplification assay to accelerate the aggregation of misfolded polypeptides present in the sample into fibrils and/or polypeptide aggregates, the presence or absence of fibrils and/or polypeptide aggregates can be detected, and the mammal can be classified as having a proteinopathy if the presence of fibrils and/or polypeptide aggregates is detected. For example, a sample obtained from a mammal can be exposed to nanoparticles (e.g., nanoparticles having a size of no more than 2 μm (e.g., no more than 1 μm) such as siNPs having a size of no more than 2 μm (e.g., no more than 1 μm)) during a seeded amplification assay to accelerate the aggregation of misfolded polypeptides present in the sample into fibrils and/or polypeptide aggregates, the presence or absence of fibrils and/or polypeptide aggregates can be detected, and the mammal can be classified as not having a proteinopathy if the absence of fibrils and polypeptide aggregates is detected.

A sample can be exposed to nanoparticles having any appropriate size (e.g., having any appropriate longest dimension such as a diameter). In some cases, a nanoparticle can have a diameter of no more than about 2 μm. For example, a nanoparticle can have a diameter of from about 2 nm to about 2 μm (e.g., from about 2 nm to about 1.8 μm, from about 2 nm to about 1.5 μm, from about 2 nm to about 1.3 μm, from about 2 nm to about 100 nm, from about 2 nm to about 75 nm, from about 2 nm to about 50 nm, from about 2 nm to about 25 nm, from about 25 nm to about 2 μm, from about 50 nm to about 2 μm, from about 75 nm to about 2 μm, from about 100 nm to about 2 μm, from about 250 nm to about 2 μm, from about 500 nm to about 2 μm, from about 750 nm to about 2 μm, from about 1 μm to about 2 μm, from about 1.3 μm to about 2 μm, from about 1.5 μm to about 2 μm, from about 18 μm to about 2 μm, from about 100 nm to about 1.75 μm, from about 250 nm to about 1.5 μm, from about 500 nm to about 1.25 μm, from about 750 nm to about 1 μm, from about 250 nm to about 750 nm, from about 500 nm to about 1 μm, or from about 750 nm to about 1.5 μm). In some cases, a nanoparticle can have a diameter of no more than about 1 μm. For example, a nanoparticle can have a diameter of from about 2 nm to about 1 μm (e.g., from about 2 nm to about 800 nm, from about 2 nm to about 600 nm, from about 2 nm to about 500 nm, from about 2 nm to about 400 nm, from about 2 nm to about 300 nm, from about 2 nm to about 200 nm, from about 2 nm to about 100 nm, from about 2 nm to about 50 nm, from about 50 nm to about 1 μm, from about 100 nm to about 1 μm, from about 200 nm to about 1 μm, from about 300 nm to about 1 μm, from about 500 nm to about 1 μm, from about 700 nm to about 1 μm, from about 900 nm to about 1 μm, from about 50 nm to about 750 nm, from about 100 nm to about 500 nm, from about 200 nm to about 400 nm, from about 50 nm to about 100 nm, from about 100 nm to about 300 nm, from about 200 nm to about 400 nm, from about 300 nm to about 500 nm, from about 400 nm to about 600 nm, from about 500 nm to about 700 nm, from about 600 nm to about 800 nm, or from about 700 nm to about 900 nm). A nanoparticle can be any shape (e.g., a sphere, a rod, a nanowire, a shell, a cube, an ellipse, and a star).

A sample can be exposed to any appropriate type of nanoparticle. A nanoparticle having a size of no more than 2 μm (e.g., no more than 1 μm) can also be referred to as a quantum dot (QD). In some cases, a nanoparticle can be a metallic nanoparticle. Examples of nanoparticles that a sample (e.g., a biological sample or an environmental sample) can be exposed to include, without limitation, gold nanoparticles (AuNPs), silver nanoparticles, platinum nanoparticles, iron nanoparticles, copper nanoparticles, dielectric nanoparticles with metallic shells, siNPs, titanium dioxide nanoparticles, tin dioxide nanoparticles, cerium oxide nanoparticles, zirconium dioxide nanoparticles, iron oxide nanoparticles, cadmium telluride QDs, and lead sulfide QDs.

In some cases, the methods and materials provided herein can include one or more nanoporous materials (e.g., one or more materials containing nanometer-scale pores) in place of or in addition to nanoparticles. For example, a sample (e.g., a biological sample or an environmental sample) can be exposed to one or more nanoporous materials in a seeded amplification assay (e.g., to accelerate the aggregation of misfolded polypeptides present in the sample into fibrils and/or polypeptide aggregates). A nanoporous material can contain pores having any appropriate size pores (e.g., can contain pores having any appropriate longest dimension such as a diameter). In some cases, a nanoporous material can contain pores that have a size (e.g., a longest dimension such as a diameter) of from 2 nm to about 200 nm (e.g., from 2 nm to about 180 nm, from 2 nm to about 150 nm, from 2 nm to about 130 nm, from 2 nm to about 100 nm, from 2 nm to about 75 nm, from 2 nm to about 50 nm, from 2 nm to about 25 nm, from 25 nm to about 200 nm, from 50 nm to about 200 nm, from 75 nm to about 200 nm, from 100 nm to about 200 nm, from 125 nm to about 200 nm, from 150 nm to about 200 nm, from 175 nm to about 200 nm, from 25 nm to about 175 nm, from 50 nm to about 150 nm, from 75 nm to about 125 nm, from 25 nm to about 75 nm, from 50 nm to about 100 nm, or from 100 nm to about 150 nm, from 125 nm to about 175 nm). In some cases, a nanoporous material can contain pores that have a size (e.g., a longest dimension such as a diameter) of from 2 nm to about 100 nm (e.g., from 2 nm to about 75 nm, from 2 nm to about 50 nm, from 2 nm to about 25 nm, from 25 nm to about 100 nm, from 50 nm to about 100 nm, from 75 nm to about 100 nm, from 25 nm to about 75 nm, from 30 nm to about 50 nm, from 40 nm to about 60 nm, from 50 nm to about 70 nm, or from 60 nm to about 80 nm). In some cases, a nanoporous material can have any appropriate size (e.g., can have any appropriate longest dimension such as a diameter), provided that the nanoporous material contains nanometer-scale pores. In some cases, a nanoporous material can be a porous oxide (e.g., a mesoporous oxide). Examples of nanoporous materials that a sample (e.g., a biological sample or an environmental sample) can be exposed to include, without limitation, porous silica (e.g., mesoporous silica), porous polytetrafluoroethylene (e.g., mesoporous polytetrafluoroethylene such mesoporous TEFLON™), zeolite particles, and metal-organic frameworks (MOFs). In some cases, a nanoporous material can be in the form of a nanoporous bead. For example, a nanoporous material can include one or more mesoporous silica beads. For example, a nanoporous material can include one or more mesoporous polytetrafluoroethylene beads (e.g., one or more TEFLON™ beads).

Any appropriate method can be used to agitate a sample (e.g., a sample suspected of containing misfolded polypeptides) in the presence of nanoparticles (e.g., nanoparticles having a size of no more than 2 μm (e.g., no more than 1 μm) such as siNPs having a size of no more than 2 μm (e.g., no more than 1 μm)). For example, a sample can be agitated by exposing the same to one or more mechanical forces. In some cases, a sample exposed to nanoparticles (e.g., nanoparticles having a size of no more than 2 μm (e.g., no more than 1 μm) such as siNPs having a size of no more than 2 μm (e.g., no more than 1 μm)) can be agitated by shaking. A sample can be shaken for any appropriate amount of time. For example, a sample can be shaken for from about 2 hours to about 96 hours (e.g., from about 2 hours to about 48 hours, from about 2 hours to about 24 hours, from about 2 hours to about 12 hours, from about 2 hours to about 8 hours, from about 8 hours to about 96 hours, from about 12 hours to about 96 hours, from about 24 hours to about 96 hours, from about 48 hours to about 96 hours, from about 12 hours to about 48 hours, from about 12 hours to about 24 hours, or from about 24 hours to about 48 hours). A sample can be shaken at any appropriate speed. For example, a sample can be shaken at from about 100 RPM to about 1200 RPM (e.g., from about 100 RPM to about 1000 RPM, from about 100 RPM to about 700 RPM, from about 100 RPM to about 500 RPM, from about 300 RPM to about 1200 RPM, from about 500 RPM to about 1200 RPM, from about 800 RPM to about 1200 RPM, from about 1000 RPM to about 1200 RPM, from about 300 RPM to about 1000 RPM, from about 500 RPM to about 800 RPM, from about 300 RPM to about 500 RPM, from about 40 RPM to about 600 RPM, from about 500 RPM to about 700 RPM, from about 600 RPM to about 800 RPM, from about 700 RPM to about 900 RPM, from about 800 RPM to about 1000 RPM, or from about 900 RPM to about 1100 RPM). A sample can be shaken at any appropriate temperature. For example, a sample can be shaken at from about 20° C. to about 60° C. (e.g., from about 20° C. to about 50° C., from about 20° C. to about 40° C., from about 20° C. to about 30° C., from about 30° C. to about 60° C., from about 40° C. to about 60° C., from about 50° C. to about 60° C., or from about 30° C. to about 50° C.).

In some cases, a sample exposed to nanoparticles (e.g., nanoparticles having a size of no more than 2 μm (e.g., no more than 1 μm) such as siNPs having a size of no more than 2 μm (e.g., no more than 1 μm)) can be agitated by sonication. A sample can be sonicated for any appropriate amount of time. For example, a sample can be sonicated for from about 5 seconds to about 30 seconds (e.g., from about 5 seconds to about 25 seconds, from about 5 seconds to about 20 seconds, from about 5 seconds to about 15 seconds, from about 5 seconds to about 10 seconds, from about 10 seconds to about 30 seconds, from about 15 seconds to about 30 seconds, from about 20 seconds to about 30 seconds, from about 25 seconds to about 30 seconds, from about 10 seconds to about 25 seconds, from about 15 seconds to about 20 seconds, from about 10 seconds to about 15 seconds, or from about 20 seconds to about 25 seconds). A sonication step can be performed any number of times. For example, a sample can be sonicated from about 2 times to about 10 times (e.g., from about 2 times to about 8 times, from about 2 times to about 6 times, from about 2 times to about 4 times, from about 4 times to about 10 times, from about 6 times to about 10 times, from about 8 times to about 10 times, from about 4 times to about 8 times, from about 4 times to about 6 times, or from about 6 times to about 8 times). In cases where a sample is sonicated two or more times, the sonication step can include a rest period between sonication cycles. A rest period can be from about 10 minutes to about 40 minutes (e.g., from about 10 minutes to about 35 minutes, from about 10 minutes to about 30 minutes, from about 10 minutes to about 25 minutes, from about 10 minutes to about 20 minutes, from about 10 minutes to about 15 minutes, from about 15 minutes to about 40 minutes, from about 20 minutes to about 40 minutes, from about 25 minutes to about 40 minutes, from about 30 minutes to about 40 minutes, from about 35 minutes to about 40 minutes, from about 15 minutes to about 35 minutes, from about 20 minutes to about 30 minutes, from about 15 minutes to about 25 minutes, or from about 25 minutes to about 35 minutes). A sample can be sonicated at any appropriate amplitude. For example, a sample can be sonicated at from about 50 watts to about 500 watts (e.g., from about 50 watts to about 300 watts, from about 50 watts to about 100 watts, from about 100 watts to about 500 watts, from about 300 watts to about 500 watts, from about 100 watts to about 300 watts, from about 100 watts to about 200 watts, from about 200 watts to about 300 watts, or from about 300 watts to about 400 watts). A sample can be sonicated at any appropriate temperature. For example, a sample can be sonicated at from about 20° C. to about 65° C. (e.g., from about 20° C. to about 50° C., from about 20° C. to about 40° C., from about 30° C. to about 65° C., from about 40° C. to about 65° C., from about 50° C. to about 65° C., or from about 30° C. to about 50° C.).

In some cases, a sample exposed to nanoparticles (e.g., nanoparticles having a size of no more than 2 μm (e.g., no more than 1 μm) such as siNPs having a size of no more than 2 μm (e.g., no more than 1 μm)) can be agitated using acoustic waves. A sample can be exposed to acoustic waves for any appropriate amount of time. For example, a sample can be exposed to acoustic waves for from about 1 hour to about 24 hours (e.g., from about 1 hour to about 24 hours, from about 1 hour to about 18 hours, from about 1 hour to about 12 hours, from about 1 hour to about 8 hours, from about 1 hour to about 4 hours, from about 8 hours to about 24 hours, from about 12 hours to about 24 hours, from about 18 hours to about 24 hours, from about 4 hours to about 18 hours, from about 8 hours to about 12 hours, from about 4 hours to about 12 hours, or from about 8 hours to about 18 hours). A sample can be exposed to acoustic waves having any appropriate frequency. For example, a sample can be exposed to acoustic waves having a frequency of from about 100 Hz to about 100,000 Hz (e.g., from about 100 Hz to about 50,000 Hz, from about 100 Hz to about 10,000 Hz, from about 100 Hz to about 1,000 Hz, from about 100 Hz to about 500 Hz, from about 500 Hz to about 100,000 Hz, from about 1,000 Hz to about 100,000 Hz, from about 10,000 Hz to about 100,000 Hz, from about 50,000 Hz to about 100,000 Hz, or from about 1,000 Hz to about 50,000 Hz).

In some cases, a sample exposed to nanoparticles (e.g., nanoparticles having a size of no more than 2 μm (e.g., no more than 1 μm) such as siNPs having a size of no more than 2 μm (e.g., no more than 1 μm)) can be agitated using light illumination. A sample can be exposed to light illumination for any appropriate amount of time. For example, a sample can be exposed to light illumination for from about 1 microsecond to about 1 hour (e.g., from about 1 nanosecond to about 1 hour, from about 1 picosecond to about 1 hour, from about 1 millisecond to about 1 hour, from about 1 second to about 1 hour, from about 1 minute to about 1 hour, from about 1 microsecond to about 1 minute, from about 1 microsecond to about 1 second, from about 1 microsecond to about 1 millisecond, from about 1 microsecond to about 1 picosecond, from about 1 microsecond to about 1 nanosecond, from about 1 nanosecond to about 1 minute, from about 1 picosecond to about 1 second, from about 1 nanosecond to about 1 picosecond, from about 1 picosecond to about 1 millisecond, from about 1 millisecond to about 1 second, or from about 1 second to about 1 minute). A light illumination step can be performed any number of times. In cases where a sample is exposed to light illumination two or more times, the light illumination step can include a rest period between light illumination cycles. A light illumination step can be performed any number of times. For example, a sample can be exposed to light illumination at any appropriate intensity. For example, a sample can be exposed to light illumination at from about 10⁵ W/m² to about 10¹⁰ W/m². Light illumination can be provided by any appropriate source. Examples of sources that can be used to provide light illumination include, without limitation, laser sources, light-emitting diodes (LEDs), lamps, and arc lamps. In some cases, light illumination can include applying heat (e.g., using an external heater). In some cases, light illumination can be used to agitate a sample (e.g., a sample suspected of containing misfolded polypeptides) in the presence of metallic nanoparticles. Seeded amplification assays that use light-based amplification (e.g., are agitated using light illumination) can be referred to as photonic seeded amplification assays such as a Photo-QuIC assay.

Any appropriate method can be used to detect the presence or absence of fibrils (e.g., fibrils formed from misfolded polypeptides). In some cases, the presence or absence of fibrils can be detected using fluorescence. For example, a sample (e.g., a biological sample or an environmental sample) can be exposed to nanoparticles (e.g., nanoparticles having a size of no more than 2 μm (e.g., no more than 1 μm) such as siNPs having a size of no more than 2 μm (e.g., no more than 1 μm)) and one or more fluorophores during a seeded amplification assay such that the presence of a fluorescent signal is indicative of the presence of fibrils. Examples of such fluorophores include, without limitation, thioflavin T (ThT), congo red, stilbene, curcumin, thiophenes, alkatrines, Chrysamine G, PIB, BF-227, and boron-dipyrromethene (e.g., BODIPY™). In some cases, a sample (e.g., a biological sample or an environmental sample) containing fibrils that was exposed to nanoparticles (e.g., nanoparticles having a size of no more than 2 μm (e.g., no more than 1 μm) such as siNPs having a size of no more than 2 μm (e.g., no more than 1 μm)) and one or more fluorophores during a seeded amplification assay can emit a fluorescent signal of from about 400 nm to about 800 nm (e.g., from about 400 nm to about 700 nm, from about 400 nm to about 600 nm, from about 400 nm to about 500 nm, from about 500 nm to about 800 nm, from about 600 nm to about 800 nm, from about 700 nm to about 800 nm, or from about 500 nm to about 700 nm). In some cases, a sample (e.g., a biological sample or an environmental sample) lacking fibrils that was exposed to nanoparticles (e.g., nanoparticles having a size of no more than 2 μm (e.g., no more than 1 μm) such as siNPs having a size of no more than 2 μm (e.g., no more than 1 μm)) and one or more fluorophores during a seeded amplification assay can emit little or no fluorescent signal (e.g., at from about 400 nm to about 800 nm).

In some cases, the methods provided herein are not antibody-based methods. For example, the methods provided herein can be performed in the absence of antibody-based techniques.

In some cases, the methods provided herein can be performed in the absence of any stimulus. For example, the methods provided herein can be performed in the absence of electrochemical stimulus.

In some cases, the methods provided herein can be performed in the absence of any sensor. For example, the methods provided herein can be performed in the absence of any colorimetric sensor. For example, the methods provided herein can be performed in the absence of any electrochemical sensor.

The methods described herein (e.g., the methods for detecting the presence or absence of a misfolded polypeptide) can be used to detect the presence or absence of any misfolded polypeptide. In some cases, a misfolded polypeptide can be associated with a disease. Examples of polypeptides that can be misfolded, and where the misfolded polypeptide can be detected as described herein include, without limitation, prion protein (PrP) polypeptides, tau polypeptides, amyloid β polypeptides, α-synuclein polypeptides, TDP-43 polypeptides, islet amyloid polypeptides (IAPPs), superoxide dismutase 1 (SOD1) polypeptides, huntingtin polypeptides, fused in sarcoma (FUS) polypeptides, and translocated in liposarcoma (TLS) polypeptides.

The methods described herein (e.g., the methods for detecting the presence or absence of a misfolded polypeptide) can be used to detect the presence or absence of a misfolded polypeptide associated with any proteinopathy. As used herein, a proteinopathy is any disease associated with misfolded polypeptides and, optionally, aggregation of one or more of the misfolded polypeptides. In some cases, a proteinopathy can be a transmissible spongiform encephalopathy (TES). In some cases, a proteinopathy can be a protein-misfolding disease (PMD). In some cases, a proteinopathy can be a tauopathy. In some cases, a proteinopathy can be an α-synucleinopathy. Examples of proteinopathies associated polypeptides that can be misfolded, and where the misfolded polypeptide can be detected as described herein include, without limitation, chronic wasting disease (CWD), Creutzfeldt-Jakob Disease, transmissible mink encephalopathy, scrapie, feline spongiform encephalopathy, ungulate spongiform encephalopathy, bovine-spongiform encephalopathy, camelid spongiform encephalopathy, Alzheimer's Disease (AD), Parkinson's Disease (PD), Pick's disease, Lewy body dementia (LBD), amyotrophic lateral sclerosis (ALS), multiple systems atrophies, progressive supranuclear palsies, corticobasal degenerations, chronic traumatic encephalopathies, and type II diabetes.

The methods described herein (e.g., the methods for detecting the presence or absence of a misfolded polypeptide) can be used to detect the presence or absence of misfolded polypeptides in any appropriate sample. In some cases, a sample can be a biological sample (e.g., a sample obtained from a mammal). In some cases, a sample can be an environmental sample. A sample can be a fresh sample or a fixed sample (e.g., a formaldehyde-fixed sample or a formalin-fixed sample). In some cases, a sample can be a processed sample. For example, a processed sample can be homogenized. For example, a processed sample can be diluted (e.g., can be diluted in a buffer such as phosphate buffered saline (PBS)).

In some cases, the methods provided herein can include isolating one or more biological molecules (e.g., polypeptides) from a sample (e.g., a biological sample or an environmental sample). For example, polypeptides can be isolated from a sample and can be enriched or concentrated prior to being amplified as described herein. In some cases, one or more biological molecules (e.g., polypeptides) isolated from a sample (e.g., a biological sample or an environmental sample) can be exposed to nanoparticles (e.g., nanoparticles having a size of no more than 2 μm (e.g., no more than 1 μm) such as siNPs having a size of no more than 2 μm (e.g., no more than 1 μm)) during a seeded amplification assay as described herein.

In some cases, the methods provided herein do not include isolating one or more biological molecules (e.g., polypeptides) from a sample (e.g., a biological sample or an environmental sample). For example, a sample can include one or more inhibitors (e.g., one or more inhibitors that can occur naturally in the sample). Examples of inhibitors that can be present in a sample (e.g., a biological sample or an environmental sample) described herein include, without limitation, mucin polypeptides, proteases, lipids (e.g., polar lipids), and hemoglobin. For example, the methods described herein (e.g., the methods for detecting the presence or absence of a misfolded polypeptide) can be used to detect the presence or absence of misfolded polypeptides in a sample that includes one or more inhibitors (e.g., one or more inhibitors that can occur naturally in the sample). In some cases, exposing a sample including one or more inhibitors (e.g., one or more inhibitors that can occur naturally in the sample) to nanoparticles (e.g., nanoparticles having a size of no more than 2 μm (e.g., no more than 1 μm) such as siNPs having a size of no more than 2 μm (e.g., no more than 1 μm)) during a seeded amplification assay can be effective to overcome the inhibitor(s) present in the sample (e.g., to improve the sensitivity of the seeded amplification assay).

In some cases, a sample (e.g., a biological sample or an environmental sample) can include less than about 20 μg of misfolded polypeptide per mL sample (μg/mL).

When the methods described herein (e.g., the methods for detecting the presence or absence of a misfolded polypeptide) are used to detect the presence or absence of misfolded polypeptides, polypeptide fibrils, and/or polypeptide aggregates (e.g., globular polypeptide aggregates) in a biological sample, the biological sample can be obtained from any appropriate mammal. In some cases, a sample can be obtained from a living mammal. In some cases, a sample can be obtained from a mammal post-mortem sample. For example, a post-mortem sample can be a mammalian tissue or byproduct intended for consumption by another mammal (e.g., a human) such as beef, mutton, lamb, or venison. In some cases, a mammal can be a cervid (e.g., can be a member of the Cervidae family). Examples of mammals that a sample can be obtained from and where the sample can be assessed for the presence or absence or misfolded polypeptides as described herein (e.g., by exposing the sample to nanoparticles (e.g., siNPs) having a size of no more than 2 μm (e.g., no more than 1 μm) during a seeded amplification assay) include, without limitation, humans, non-human primates (e.g., monkeys), camels, mink, cats, canines, cattle, sheep, mice, rats, hamsters, brocket, chital, elk, fallow deer, marsh deer, mule deer, muntjac, moose, pampas deer, red deer, reindeer, roe deer, sambar deer, sika, white-tailed deer, antelope, and goats.

When the methods described herein (e.g., the methods for detecting the presence or absence of a misfolded polypeptide) are used to detect the presence or absence of misfolded polypeptides, polypeptide fibrils, and/or polypeptide aggregates (e.g., globular polypeptide aggregates) in a biological sample, the biological sample can be any type of biological sample. In some cases, a biological sample can be a secretion (e.g., a bodily secretion). Examples of biological samples that can be assessed for the presence or absence or misfolded polypeptides as described herein (e.g., by exposing the sample to nanoparticles (e.g., siNPs) having a size of no more than 2 μm (e.g., no more than 1 μm) during a seeded amplification assay) include, without limitation, lymph tissue, muscle tissue, tonsil tissue, skin tissue, brain tissue, brain-stem tissue, blood (e.g., whole blood, serum, and plasma), cerebrospinal fluid, urine, feces, saliva, mucus, liver tissue, heart tissue, intestinal tissue, semen, spleen tissue, and eye tissue.

When the methods described herein (e.g., the methods for detecting the presence or absence of a misfolded polypeptide) are used to detect the presence or absence of misfolded polypeptides in an environmental sample, the environmental sample can be obtained from any appropriate source. Examples of sources that a sample can be obtained from and where the sample can be assessed for the presence or absence of misfolded polypeptides as described herein (e.g., by exposing the sample to nanoparticles (e.g., siNPs) having a size of no more than 2 μm (e.g., no more than 1 μm) during a seeded amplification assay) include, without limitation, soil, water, dust, and plants.

When the methods described herein (e.g., the methods for detecting the presence or absence of a misfolded polypeptide) are used to detect the presence or absence of misfolded polypeptides in an environmental sample, the environmental sample can be obtained by any appropriate method. Examples of methods that can be used to obtain an environmental sample that can be assessed for the presence or absence of misfolded polypeptides as described herein (e.g., by exposing the sample to nanoparticles (e.g., siNPs) having a size of no more than 2 μm (e.g., no more than 1 μm) during a seeded amplification assay) include, without limitation, swabs and filters (e.g., air filtration system filters).

When the methods described herein (e.g., the methods for detecting the presence or absence of a misfolded polypeptide) are used to detect the presence or absence of misfolded polypeptides in an environmental sample, the environmental sample can be obtained from any environmental setting. Examples of environmental settings that an environmental sample that can be assessed for the presence or absence of misfolded polypeptides as described herein (e.g., by exposing the sample to nanoparticles (e.g., siNPs) having a size of no more than 2 μm (e.g., no more than 1 μm) during a seeded amplification assay) can be obtained from include, without limitation, natural habitats, waterways, farms, food processing facilities (e.g., meat processing facilities), medical facilities, animal feed processing facilities, water-treatment facilities, and medical facilities (e.g., medical clinics, emergency rooms, urgent cares, and hospitals such as human hospitals and veterinary hospitals). When an environmental sample is obtained from a food processing facility, the food processing facility can process food intended for mammalian (e.g., human) consumption. For example, an environmental sample can be obtained from a food processing facility that processes agricultural commodities (e.g., alfalfa, corn, beets, soybeans, oats, grasses, potatoes, straw, and related byproducts) for mammalian consumption. When an environmental sample is obtained from a medical facility, the sample can be obtained from any surface (e.g., a stainless steel surface) in frequent contact with patients and/or biological fluids (e.g., blood, urine, and feces). For example, an environmental sample obtained from a medical facility can be obtained from surgical tools, examination surfaces, and countertops.

In some cases, the presence or absence of misfolded polypeptides in a sample can be confirmed using one or more techniques traditionally used to detect the presence of the presence or absence of misfolded polypeptides in a sample. For example, enzyme-linked immunosorbent assay (ELISA), immunohistochemistry (IHC), and/or RT-QuIC tests can be used to confirm the detection of the presence or absence of misfolded polypeptides in a sample.

This document also provides methods and materials for treating a mammal (e.g., a human) identified as having a proteinopathy as described herein (e.g., based, at least in part, on the presence of fibrils formed from misfolded polypeptides). For example, a mammal identified as having a proteinopathy based, at least in part, on the presence of fibrils formed from misfolded polypeptides in a sample obtained from the mammal can be subjected to one or more (e.g., one, two, three, four, five or more) therapies that can be used to treat one or more symptoms of a proteinopathy. Examples of therapies that can be used to treat one or more symptoms of a proteinopathy include, without limitation, physical therapy, occupational therapy, speech therapy, electro stimulation, and any combinations thereof.

In some cases, the methods and materials provided herein can be used to select a non-human mammal for treatment. For example, a non-human identified as having a proteinopathy based, at least in part, on the presence of fibrils formed from misfolded polypeptides in a sample obtained from the mammal can be selected for isolation. In some cases, a non-human identified as having a proteinopathy based, at least in part, on the presence of fibrils formed from misfolded polypeptides in a sample obtained from the mammal can be isolated from other mammals. For example, a non-human identified as having a proteinopathy based, at least in part, on the presence of fibrils formed from misfolded polypeptides in a sample obtained from the mammal can be selected for euthanization. In some cases, a non-human identified as having a proteinopathy based, at least in part, on the presence of fibrils formed from misfolded polypeptides in a sample obtained from the mammal can be euthanized.

In some cases, the methods and materials provided herein can be used to select a facility for treatment. For example, a facility identified as having the presence of one or more misfolded polypeptides in a sample (e.g., an environmental sample) obtained from the facility can be selected for one or more (e.g., one, two, three, four, five or more) treatments that can be used to sterilize the facility. In some cases, a facility identified as having the presence of one or more misfolded polypeptides in a sample (e.g., an environmental sample) obtained from the facility can be sterilized.

The invention will be further described in the following examples, which do not limit the scope of the invention described in the claims.

### EXAMPLES

### Example 1: Nanoparticle-Enhanced RT-QuIC Diagnostic Assay

This Example describes the discovery that nanoparticles can increase RT-QuIC sensitivity. For example, the addition of silica nanoparticles to RT-QuIC experiments (defined as Nano-QuIC) for CWD diagnostics greatly improved performance, halving the time to disease detection and overcoming inhibitor effects thus increasing sensitivity, e.g., by 10-fold.

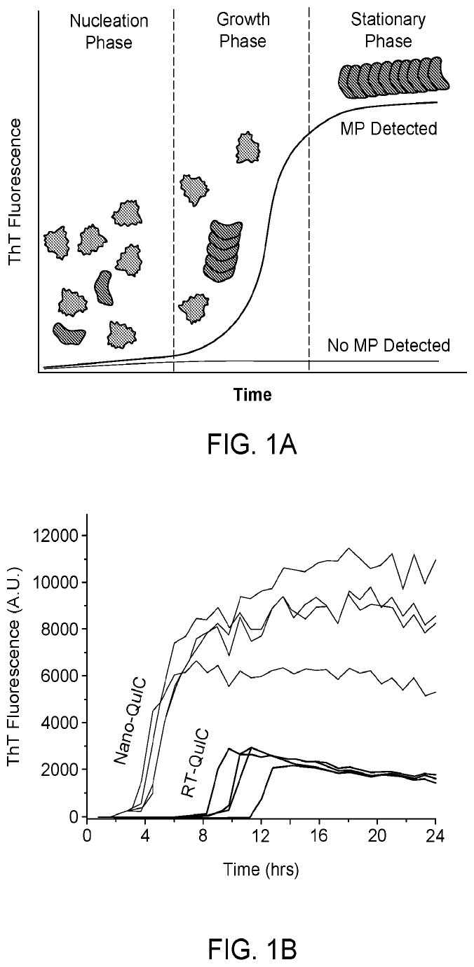

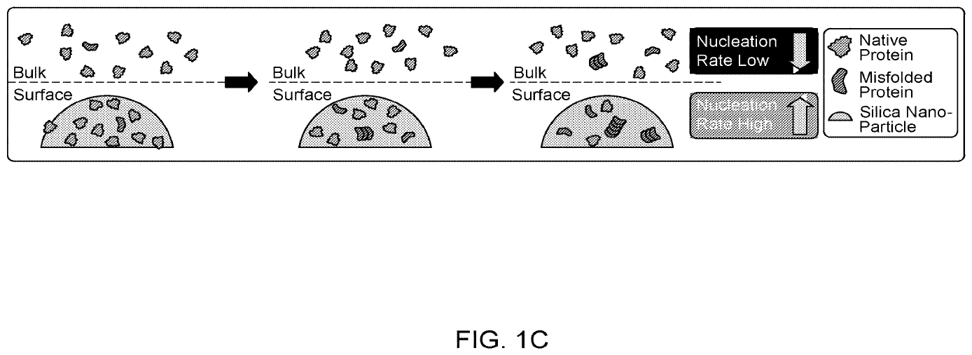

Silica NPs (siNPs) were applied as reagents in RT-QuIC reactions to examine their impact on the detection of CWD prions in lymphoid tissues of wild white-tailed deer. All experiments were performed with recombinant hamster PrP (rHaPrP) as the RT-QuIC reaction substrate. It was observed that RT-QuIC performance was significantly improved in the presence of siNPs, corresponding to siNP size. optimal RT-QuIC conditions represented by the highest rate of amyloid formation (RAF; FIG. 1B) and lowest false-positivity rate were identified by exploring various combinations of NP diameters and concentrations as well as reaction temperatures. It was found that the addition of 50 nm siNPs optimized the performance of RT-QuIC by significantly increasing both the RAF and ThT fluorescence (FIG. 1B).

Sigmoidal-like curves were used to fit the kinetic fluorescent data and extract key parameters that describe the lag time of the nucleation phase and the time constant of the growth phase. This information revealed that much of the speed increase was likely due to a higher nucleation rate of MP on the surface of the siNP compared to the nucleation rate of the bulk solution (FIG. 1C). Additionally, the accelerating effects of NPs were not just limited to siNPs but can also occurred by adding gold nanoparticles (AuNPs) to the RT-QuIC reaction. Finally, to test whether RT-QuIC with siNPs could overcome effects of inhibitors, CWD-positive lymphoid tissues were serially diluted and it was shown that NP-enhance RT-QuIC (Nano-QuIC) can readily detect the presence of CWD prions at a concentration that had not been achieved before.

### Results

Diameter and Concentration Effects of Silica NPs (siNPs)

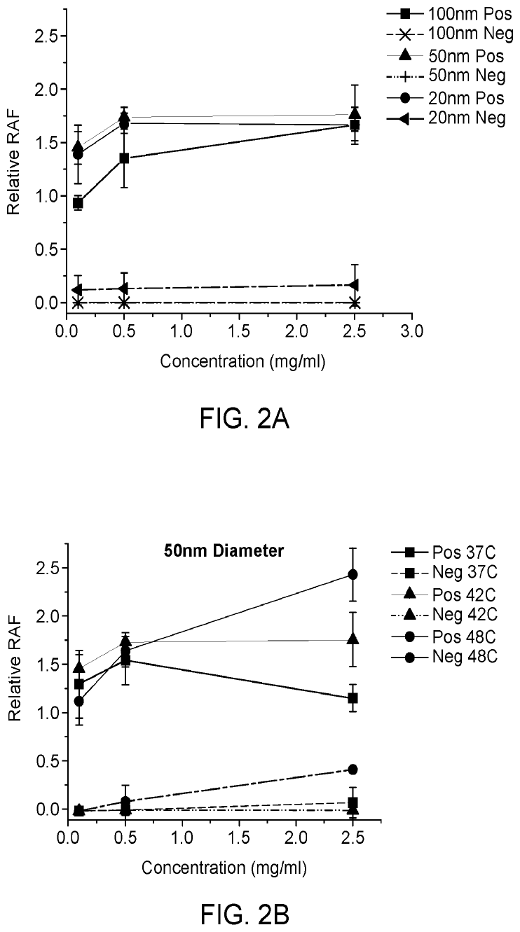

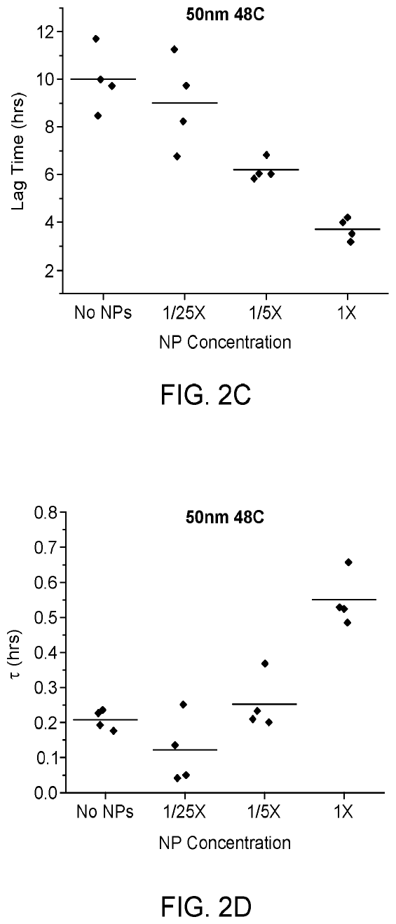

To examine the effects of nanoparticles on RT-QuIC performance, siNPs ranging from 20 nm-100 nm were added as a reagent to RT-QuIC reactions seeded with CWD positive or CWD negative tissue homogenates (parotid lymph nodes). Concentrations of siNPs were 0.1 mg/mL, 0.5 mg/mL, and 2.5 mg/mL (referred to as 1/25×, ⅕× and 1× respectively). Reactions were run for 48 hours at a temperature of 42° C. All diameters of siNPs examined herein affected the RAF when compared to the RAF of reactions having no siNPs (FIG. 2A). The ratio between the RAF of reactions with and without siNPs gives a parameter known as the relative RAF. For all siNP diameters, the relative RAF increased as the concentration of the siNPs increased (FIG. 2A). This led to an average time to CWD detection of 6.3 hours (95% confidence interval [CI]: ±0.96 hours) for 50 nm siNPs at 42° C., 4.9 hours faster than traditional RT-QuIC reactions of the same sample (average time to detection of 11.2 hours (95% CI: ±1.00 hour)). Importantly, the optimal siNP reaction parameters for diagnostic assessment of PrP^{CWD} positive tissues examined herein (50 nm siNPs at 42° C.) yielded no false positives.

### Temperature Effects

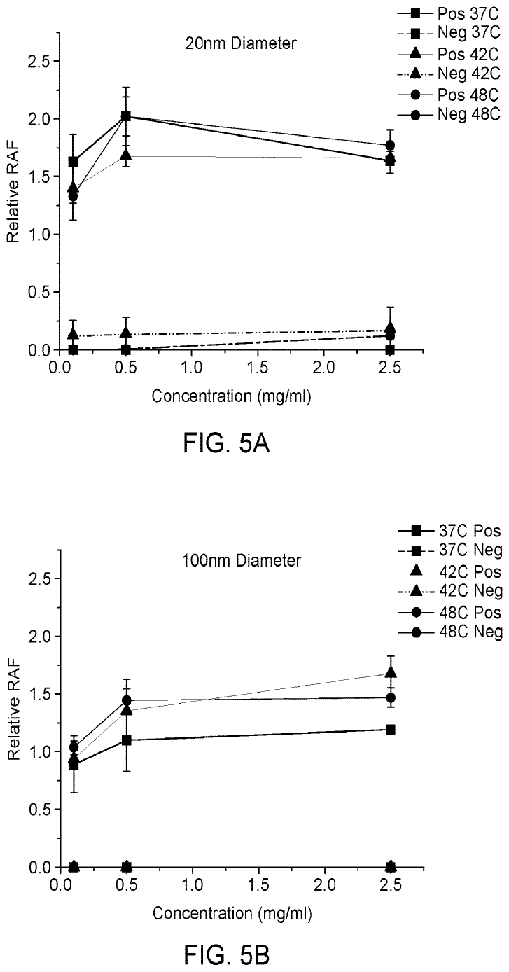

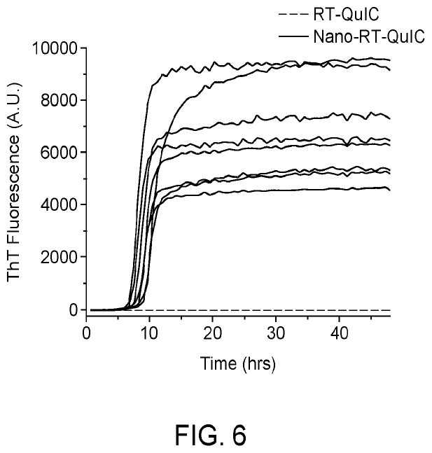

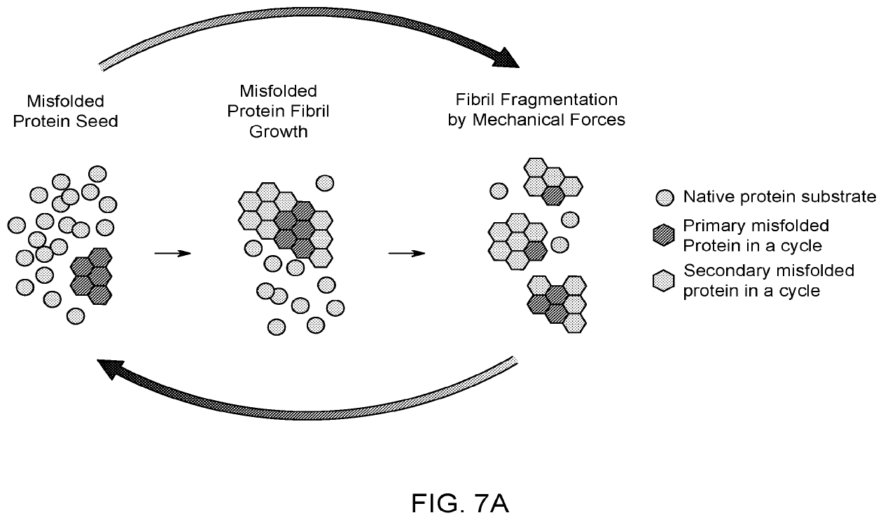

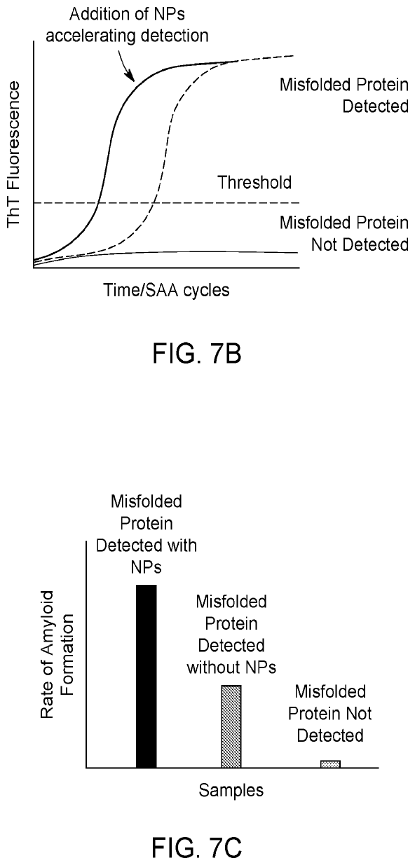

The temperature of a particular RT-QuIC experiment directly influences diagnostic sensitivity and specificity. Higher temperatures typically yield higher RAFs for a given true-positive diagnostic sample; however, such conditions increase the risk of spontaneous misfolding of the substrate protein. Lower temperatures give lower RAF values, extending diagnostic time and potentially leading to false-negative results, but decreasing false positivity rates. To identify optimal conditions that maximize Nano-QuIC sensitivity and specificity, experiments were performed at 37° C., 42° C., and 48° C. for 20 nm-100 nm siNPs with concentrations ranging from 0.1-2.5 mg/mL. Once again, the relative RAF was found by comparing reactions with and without siNPs. For reactions at 37° C., all diameters of siNPs led to higher relative RAFs of CWD-positive samples vs. the same samples tested with traditional RT-QuIC (FIG. 2B, FIG. 5A, and FIG. 5B). However, compared to siNP experiments performed at higher temperatures, CWD-positive reactions performed at 37° C. exhibited slower RAFs. For Nano-QuIC experiments performed at 48° C., all diameters of siNPs again led to a higher relative RAF (FIG. 2B, FIG. 5A, and FIG. 5B). Additionally, experiments performed at 48° C. had shorter times to detection (higher raw RAFs) compared to reactions at lower temperatures. Nano-QuIC performed using 50 nm siNPs at 2.5 mg/mL and a temperature of 48° C. gave the largest relative RAF and the fastest time to detection, just 4.1 hours (95% CI: ±0.45 hours) compared to 10.1 hours (95% CI: ±1.44 hours) for traditional RT-QuIC, a 2.5× improvement (FIG. 2B). No false positive replicates were observed across our CWD-negative samples (see discussion). Using these parameters, Nano-QuIC was performed on a blinded set of 10 CWD positive and 10 CWD negative wild white-tailed deer retropharyngeal lymph nodes and all tissues were classified with 100% sensitivity and specificity (Table 1).

TABLE 1
Blinded sample set of 10 CWD Positive and 10 CWD negative
retropharyngeal lymph nodes. Nano-QuIC conditions 50
nm siNPs, 48° C. Sample was considered positive
if 50% or more of its replicates were positive.

| Process ID | ELISA/IHC classification | Number of positive replicates |
|---|---|---|
| 1378 | − | 0 |
| 922 | + | 4 |
| 921 | + | 4 |
| 1394 | − | 1 |
| 1384 | − | 0 |
| 926 | + | 4 |
| 1392 | − | 0 |
| 923 | + | 2 |
| 1376 | + | 4 |
| 927 | + | 4 |
| 1389 | − | 0 |
| 1382 | − | 0 |
| 1395 | − | 0 |
| 925 | + | 4 |
| 958 | − | 0 |
| 1383 | − | 0 |
| 929 | + | 4 |
| 914 | + | 4 |
| 928 | + | 4 |
| 1379 | − | 1 |

### Aggregation Kinetics and Mechanism

The formation of proteinaceous fibrils is a process whereby misfolded polypeptides interact with one another to form large linear structures. Modeling fibril formation in RT-QuIC is achieved using three phases: the nucleation (lag) phase, the growth (elongation) phase and the stationary phase (FIG. 1A). During the nucleation phase, native polypeptides misfold into nuclei units. Because ThT fluoresces when bound to fibrils and not single monomers, the nucleation phase was characterized by zero or low fluorescence values. In the elongation phase, nuclei act as templates to efficiently misfold other native polypeptides, ultimately producing linear fibrils. In the presence of mechanical shaking or sonication, these fibrils can break, thus creating additional nuclei for native polypeptides to misfold and making more fibrils. The elongation phase was characterized by exponential growth of fibrils as documented by exponential growth of ThT fluorescence (FIG. 1A and FIG. 2B). In the final phase, the stationary phase, ThT fluorescence ceases to increase exponentially and stabilizes. This model accounted for both the propagation of infectious prions observed in transmissible spongiform encephalopathies (e.g., CWD or Creutzfeldt-Jakob disease) as well as the misfolding and spread of polypeptides associated with numerous neurodegenerative diseases (e.g., alpha synuclein in Parkinson's, TDP-43 in ALS, and tau in Alzheimer's disease).

The three phases of protein amplification using RT-QuIC were modeled using sigmoidal-like curves.

$$Y = {y}_{i} + {m}_{i} x + \frac{{y}_{f} + {m}_{f} x}{1 + {e}^{- [ ( x - {x}_{0} ) / τ ]}} \qquad ( eq 1 )$$

where yᵢ and mᵢ are associated with the initial ThT fluorescence and slope of the nucleation phase. y_{f} and m_{f} are associated with the final ThT fluorescence and slope of the stationary phase. The time to 50% of the max ThT fluorescence is λₒ and τ is the time constant for fibril growth. The length of the nucleation phase, which is approximately the time to detection, is given by the lag time xₒ−2τ.

To characterize the kinetics of how siNPs influence RT-QuIC reactions (50 nm siNPs at 48° C.), Equation 1 was used to develop fits that yielded semi-quantitative values of lag time and the time constant for fibril growth (τ) for concentrations 1/25×-1× (See supporting methods). It was observed that as the concentration of siNPs increased, the lag time decreased (FIG. 2C) thus indicating that the siNPs can play a crucial role in the formation of the initial nuclei for fibrillation.

At pH 7.4, the rHaPrP, used as the RT-QuIC substrate, had a net positive charge whereas the siNPs had a net negative charge leading to a force of attraction. This likely promoted the absorption of rHaPrP onto the surface of the silica NPs, thus increasing the local effective concentration of RT-QuIC substrate. Additionally, infectious prions can absorb onto glass and silica surfaces. Absorbed polypeptides were not stationary at one location on a given siNP, but can diffuse across the surface and interact with one another. Because the local concentration of polypeptides on the siNP surface was higher than in the bulk reaction space, there was more opportunity for RT-QuIC substrate to interact with misfolded protein seeds, thus directly influencing reaction kinetics by facilitating a more efficient nucleation phase. Additionally, polypeptides absorbed onto surfaces often change their conformation. These changes in conformation could make rHaPrP more susceptible to misfolding in the presence of prion seeds, thus increasing the nucleation rate. The three characteristic phases of protein amplification (discussed above) were entirely absent from negative samples.

The time constant for fibril growth (t) was used to compare fibril growth phases between different RT-QuIC reaction conditions. For 50 nm siNPs at 48° C., it was observed that as the concentration of nanoparticles increased, the growth phase was characterized by a longer τ (FIG. 2D). Additionally, the maximum ThT fluorescence of solutions with NPs were larger than solutions without NPs (FIG. 2B). This observation might be accounted for if the siNPs, in the presence of mechanical shaking, were more efficiently breaking the fibrils into smaller units. A greater number of fibril nuclei can recruit more haPrP substrate, thus contributing to an exponential growth phase. Such a mechanism can effectively account for the unique sigmoidal curves observed in Nano-QuIC.

### Gold Nanoparticles

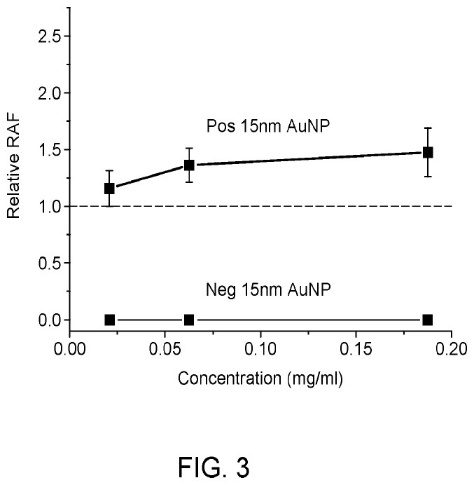

To demonstrate that acceleration effects of NPs on RT-QuIC performance can apply to other NP types, additional experiments were conducted using AuNPs. 15 nm citrate-capped AuNPs were added as reagents to RT-QuIC reactions to obtain final concentrations of 20.75 μg/mL, 62.5 μg/mL, or 187.5 μg/mL AuNPs. CWD-positive or CWD-negative tissue homogenates were added to the solutions and RT-QuIC reactions ran for 48 hours at 42° C. RAFs were higher for all solutions containing AuNPs compared to RAFs of reactions without AuNPs (FIG. 3). The fastest average time to detection was found to be 8.2 hours (95% CI: ±1.21 hours) compared to the average time of detection of the no AuNP solutions: 12.5 hours (95% CI: ±1.26 hours).

### Overcoming RT-QuIC Inhibitors

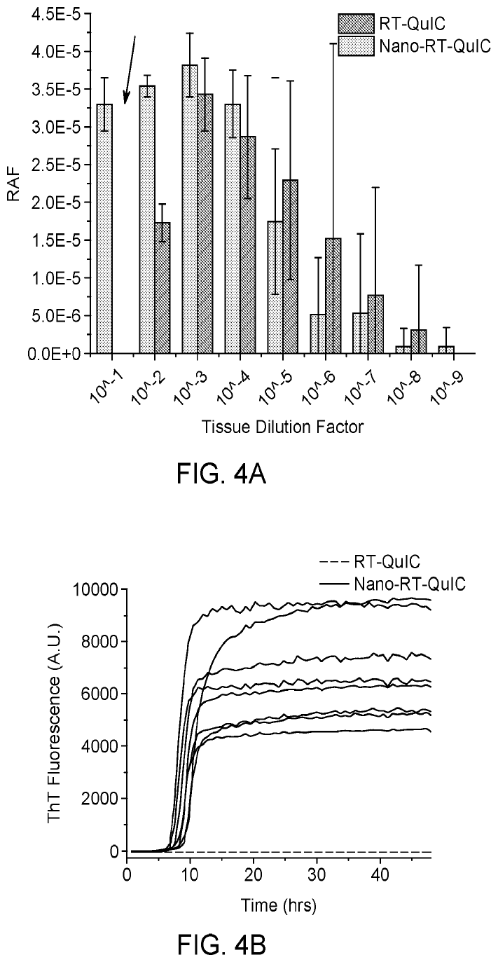

Tissue dilution series experiments were performed for 50 nm siNPs at 42° C. to compare the sensitivity of Nano-QuIC and RT-QuIC. Ten-fold dilutions of CWD-positive tissue seeds were created from 10⁻¹ to 10⁻⁹. Subsamples of these dilutions (i.e., seeds) were then added to Nano-QuIC and traditional RT-QuIC reactions. Nano-QuIC and RT-QuIC detected seeding activity in reactions with seeds diluted to 10⁻⁹ and 10⁻⁸, respectively, documenting the diagnostic sensitivity of these assays. However, for less dilute seeds, Nano-QuIC greatly outperformed traditional RT-QuIC. At dilutions of 10⁻¹ PrP^{CWD} positive tissue, traditional RT-QuIC exhibited no seeding activity, whereas Nano-QuIC clearly detected the presence of Prp^{CWD} as reflected by high RAF values (FIGS. 4A and 4B). Moreover, for Prp^{CWD} positive tissue dilutions of 10⁻², Nano-QuIC yielded double the RAF compared to traditional RT-QuIC (FIG. 4A).

Biological samples, especially from clinical settings, are extremely complex thus making diagnostics challenging. Similar to inhibitors that negatively impact PCR performance, RT-QuIC is susceptible to inhibitors that hamper detection and/or the misfolding of protein substrates. RT-QuIC inhibitors can include polypeptides of the mucin family and polar lipids, however, it is likely that a variety of additional inhibitory factors exist. Traditionally, RT-QuIC inhibitors are overcome by diluting tissue samples, and concordantly reaction limiting compounds, until diagnostic sensitivity performs as expected for true-positive samples. However, both inter- and intra-individual MP heterogeneity, as well as stage of neurodegenerative disease (i.e., early vs. late stages), directly influence the quantity of MPs present within a given biological sample. Therefore, the practice of diluting diagnostic samples to overcome inhibitors might contribute to the production of false-negatives (i.e., the diagnostic samples are true positives yet have very low levels of MPs that are subsequently diluted beyond detection). These data indicate that Nano-QuIC has the potential to overcome false-negatives associated with sample dilution and/or inhibitors as the siNP-improved assay exhibited 100% sensitivity in replicates where traditional RT-QuIC failed (FIG. 3). Without being bound by theory, it is believed that the protein substrate (rHaPrP) can bind to the surface of the siNPs, thus increasing local rHaPrP concentration and allowing for more efficient interaction with MPs and subsequent misfolding, thereby overcoming inhibitors and ultimately improving diagnostic sensitivity.

Together, these results demonstrate that incorporating siNPs as a reagent to RT-QuIC reactions can improve diagnostic performance.

### Methods

### Tissue Preparation

A CWD positive and a CWD negative parotid lymph node from white tailed deer (WTD) were selected as tissues for this study. Parotid lymph nodes were classified using Bio-Rad TeSeE Short Assay Protocol (SAP) Combo Kit (BioRad Laboratories Inc., Hercules, CA, USA). Tissues were homogenized in PBS (10% w:v) in 2 mL tubes containing 1.5 mm zirconium beads with a BeadBug Homogenizer (Benchmark Scientific, Sayreville New Jersey, USA) on max speed for 90 seconds. Samples were aliquoted and frozen. These samples were referred to as 10% homogenates. For this study the same homogenates were used for all experiments except for the blinded samples set. All CWD positive and negative samples were selected based on independent ELISA, IHC, and/or RT-QuIC results and were subsampled as described in Schwabenlander et al. (Schwabenlander et al., J. Wildl. Dis., 58 (1): 50-62 (2021)).

### Preparation of Recombinant Substrate.

Recombinant PrP (rPrP) production and purification were done as described in elsewhere (Schwabenlander et al., J. Wildl. Dis., 58 (1): 50-62 (2021)). The substrate was derived from a truncated form (amino acids 90-231) of the Syrian hamster PRNP gene cloned into pD431—SR (ATUM, Newark, CA, USA) and was expressed in Rosetta (DE3) E. coli (Sigma-Aldrich, St. Louis, MO, USA). Recombinant hamster PrP was used in this study due to its seemingly universal proclivity to misfold in the presence of infectious prions for a large variety of species.

### QuIC for Tissues.

For Nano-QuIC analysis, a master mix was made to the following specifications: 1×PBS, 1 mM Ethylenediaminetetraacetic acid (EDTA), 170 mM NaCl, 10 μM thioflavin T (ThT), 0.1 mg/mL recombinant hamster PrP substrate (rPrP) and siNPs (nanoComposix a Fortis Life Sciences Company, San Diego, CA, USA) with final well concentrations of 2.5 mg/mL, 0.5 mg/mL, 0.1 mg/mL, or no NPs. For AuNP reactions, 15 nm citrate capped AuNPs (Nanopartz, Loveland, CO, USA) were added to master mix solutions with final well concentrations of 20.8 μg/mL, 62.5 μg/mL and 187.5 μg/mL. For RT-QuIC reactions, the master mix was the same except with no NPs. For temperature and concentration experiments, the 10% tissue homogenates (prepared as described above) were further diluted 100-fold in dilution buffer (0.1% Sodium Dodecyl Sulfate (SDS), 1×PBS, 1×N−2 supplement (μL of N2 per 100 μL SDS/PBS) (Thermofisher Scientific, Waltham, MA, USA)). For dilution experiments (FIGS. 4A and 4B) 10-fold serial dilutions were made from the 10% homogenate (e.g., 10 μL of 10% homogenate in 90 μL of dilution buffer is 10⁻¹ dilution, etc). 2 μL of the diluent were added to each well containing 98 μL of master mix. For temperature and concentration experiments, four replicates were used for each condition. For dilution experiments, eight replicates were used. Plates for Nano-QuIC were amplified for 48 hours on a FLUOstar® Omega plate reader (BMG Labtech, Cary, North Carolina, USA; various temperatures, 700 rpm, double orbital, shake for 57 seconds, rest for 83 seconds). Fluorescent readings were taken in 45-minute intervals. Following methods from Rowden et al. (Rowden et al., Pathogens, 12 (2): 309 (2023)), RAF was determined as the inverse seconds (s⁻¹) required for curves to surpass a threshold of twice the background fluorescent signal at cycle 4 of the reaction.

### Blinded Sample Set

A 10 CWD negative and 10 CWD positive retropharyngeal lymph nodes were prepared in the same way as the parotid lymph nodes above. Samples were blinded and ran using both RT-QuIC and Nano-QuIC at 48° C. SiNPs used were 50 nm at a concentration of 2.5 mg/mL. Samples were classified as positive if 2 or more of the 4 replicates were positive using maxpoint methods (Rowden et al., Pathogens, 12 (2): 309 (2023)).

### Evaluation of Kinetics Data

ThT kinetic curves were modeled as having a nucleation, growth, and stationary phase. The maximum change in fluorescence between time points was found. To better fit the region of interest (nucleation and growth phase), data was used from the beginning of the nucleation phase to 10 points after the maximum change in fluorescence. This captured the growth phase and the beginning region of the stationary phase. Fits of Eq 1 were made using Origin version 9.9.5. Average R² values and corresponding standard deviation can be seen in Table 2.

TABLE 2
R² values of the fits in FIGS. 3C and 3D.

| Sample | Average R² | Standard Deviation |
|---|---|---|
| 1X 50 nm NPs 48 C. | 0.99012 | 0.00550 |
| 1/5X 50 nm NPs 48 C. | 0.98513 | 0.01514 |
| 1/25X 50 nm NPs 48 C. | 0.99061 | 0.00155 |
| Pos Cont No NPs 48 C. | 0.99838 | 0.00099 |

### Example 2: Nanoparticle-Enhanced RT-QuIC Diagnostic Assay

### Methods

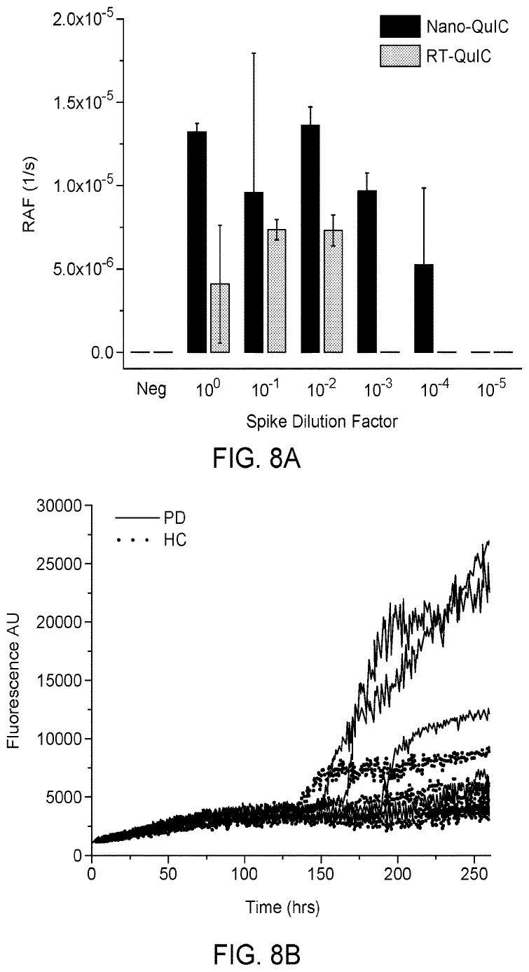

For artificial spiking of human plasma experiments (FIG. 8A), EDTA plasma samples were obtained. Master mixes were prepared as to the following specifications: 1× phosphate buffer (9.2 mM NaH₂PO₄, 2.8 mM Na₂HPO₄, 2.7 mM KCl, and 137 mM NaCl), 1 mM ethylenediaminetetraacetic acid (EDTA), 170 mM NaCl (in addition to that in the NaCl in the phosphate buffer), 10 μM thioflavin T (ThT), and 0.09 mg/mL recombinant human alpha synuclein. The pH was approximately 6.3. For Nano-QuIC reactions, there was a concentration of 2.5 mg/mL 50 nm silica nanoparticles. 10 μL of spontaneously misfolded α-synuclein (0.09 mg/mL) was sonicated (200 W 15 seconds on, 15 seconds off, 3× times) and spiked into 90 μL of human plasma, this solution is referred to as 10° dilution (FIG. 8A). This solution was then serially diluted ten-fold in human plasma down to a final spike concentration of 90 μg/mL (referred to as 10⁻⁵). Each spike dilution was then diluted one hundred-fold in SDS/PBS (0.1% sodium dodecyl sulfate (SDS), 1×PBS). 2 μL of this final dilution was then added to 98 μL of the master mix in a 96 well plate. Plates were put onto a plate reader (BMG Labtech, Cary, North Carolina, USA); 700 rpm, double orbital, shake for 60 seconds, rest for 60 seconds) at 42° C. for at least 48 hours.

For human plasma sample testing (FIG. 8B), plasma samples from subjects diagnosed with Parkinson's disease and not diagnosed were obtained. 200 μl of human plasma was centrifuged at 21,000 g for 40 minutes. 185 μl of supernatant was removed and the remaining liquid/pellet was resuspended in 20 ul 0.1% SDS in 1×PBS. 2 ul of this mixture was put into a master mix containing Silica nanoparticles (see above) of 98 μl on a 96 well plate and 1 ul of 0.1% SDS was added. Samples were put onto a BMG plate reader at 700 rpm, 42 C, 60 sec on/off interval for 260 hours

Together, these results demonstrate that Nano-QuIC can be used to overcome inhibitory barriers commonly associated with blood/plasma samples in conventional RT-QuIC assays. Whole blood samples are extremely complex and challenging to diagnostics. In samples such as CSF, blood contamination is thought to greatly interfere with QuIC diagnostics. Even in the presence robust inhibitors present in human plasma, Nano-QuIC outperformed RT-QuIC, having no false positive replicates while detecting misfolded α-synuclein at a level 900 μg/mL, a 100-fold lower than RT-QuIC (FIG. 8A).

In addition, pooled human plasma from patients diagnosed with Parkinson's disease versus not diagnosed was tested. It was observed that the Parkinson's samples showed higher seeding activity (FIG. 8B). The seeding activity for this reaction took ~160 hours to observe which was longer than needed for the spiking plasma experiments in FIG. 8A; however, activity is still present. These data support the use of Nano-QuIC for human disease diagnostics. Longer seeding times may be due to an extremely low (perhaps sub pg) quantity of misfolded α-synuclein in the plasma combined with a high concentration of inhibitors.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for detecting the presence or absence of misfolded polypeptides in a sample, wherein said method comprises:
(a) exposing said sample to (i) nanoparticles having a size of no more than 1 μm or (ii) a nanoporous material having a pore size of from 2 nm to about 200 nm, during a seeded amplification assay, wherein misfolded polypeptides, when present within said sample, form fibrils;
(b) detecting said fibrils if formed in step (a);
(c) identifying said sample as having said presence of said misfolded polypeptides if said fibrils are detected; and
(d) identifying said sample as lacking said misfolded polypeptides if said fibrils are not detected.

2. The method of claim 1, wherein said sample is a biological sample.

3. The method of claim 2, wherein said biological sample is obtained from a living mammal.

4-5. (canceled)

6. The method of claim 2, wherein said biological sample is obtained from a mammal post-mortem.

7. (canceled)

8. The method of claim 1, wherein said sample is an environmental sample.

9-13. (canceled)

14. The method of claim 1, wherein said method comprises, prior to said step (a), isolating polypeptides from said sample.

15-18. (canceled)

19. The method of claim 1, wherein said seeded amplification assay is completed in less than about 96 hours.

20. (canceled)

21. A method for detecting the presence or absence of misfolded polypeptides in a sample, wherein said method comprises:
(a) exposing said sample to (i) nanoparticles having a size of no more than 1 μm or (ii) a nanoporous material having a pore size of from 2 nm to about 200 nm, during a seeded amplification assay, wherein misfolded polypeptides, when present within said sample, form globular polypeptide aggregates;
(b) detecting said globular polypeptide aggregates if formed in step (a);
(c) identifying said sample as having said presence of said misfolded polypeptides if said globular polypeptide aggregates are detected; and
(d) identifying said sample as lacking said misfolded polypeptides if said globular polypeptide aggregates are not detected.

22. The method of claim 21, wherein said sample is a biological sample.

23. The method of claim 22, wherein said biological sample is obtained from a living mammal.

24-25. (canceled)

26. The method of claim 22, wherein said biological sample is obtained from a mammal post-mortem.

27. (canceled)

28. The method of claim 21, wherein said sample is an environmental sample.

29-33. (canceled)

34. The method of claim 21, wherein said method comprises, prior to said step (a), isolating polypeptides from said sample.

35-38. (canceled)

39. The method of claim 21, wherein said seeded amplification assay is completed in less than about 96 hours.

40. (canceled)

41. A composition comprising (i) nanoparticles having a size of no more than 1 μm or (ii) a nanoporous material having a pore size of from 2 nm to about 100 nm, for use in accelerating a seeded amplification assay.

42. (canceled)

43. The composition of claim 41, wherein said composition comprises nanoparticles selected from the group consisting of silica nanoparticles, gold nanoparticles, silver nanoparticles, platinum nanoparticles, iron nanoparticles, copper nanoparticles, dielectric nanoparticles with metallic shells, titanium dioxide nanoparticles, tin dioxide nanoparticles, cerium oxide nanoparticles, zirconium dioxide nanoparticles, iron oxide nanoparticles, cadmium telluride quantum dots, and lead sulfide quantum dots.

44-86. (canceled)

87. The composition of claim 41, wherein said composition comprises nanoporous material is selected from the group consisting of mesoporous silica, mesoporous polytetrafluoroethylene, zeolite particles, and MOFs.

88. (canceled)

89. A method for detecting the presence or absence of misfolded polypeptides in a sample, wherein said method comprises:
(a1) exposing said sample to nanoparticles having a size of no more than 1 μm during a seeded amplification assay, wherein said seeded amplification assay comprises exposing said sample to acoustic waves, wherein misfolded polypeptides, when present within said sample, form fibrils;
(b1) detecting said fibrils if formed in step (a);
(c1) identifying said sample as having said presence of said misfolded polypeptides if said fibrils are detected; and
(d1) identifying said sample as lacking said misfolded polypeptides if said fibrils are not detected; or
wherein said method comprises:
(a2) exposing said sample to nanoparticles having a size of no more than 1 μm during a seeded amplification assay, wherein said seeded amplification assay comprises exposing said sample to acoustic waves, wherein misfolded polypeptides, when present within said sample, form globular polypeptide aggregates;
(b2) detecting said globular polypeptide aggregates if formed in step (a);
(c2) identifying said sample as having said presence of said misfolded polypeptides if said globular polypeptide aggregates are detected; and
(d2) identifying said sample as lacking said misfolded polypeptides if said globular polypeptide aggregates are not detected; or
wherein said method comprises:
(a3) exposing said sample to metallic nanoparticles having a size of no more than 1 μm during a seeded amplification assay, wherein said seeded amplification assay comprises exposing said sample light illumination of said sample, wherein misfolded polypeptides, when present within said sample, form fibrils;
(b3) detecting said fibrils if formed in step (a);
(c3) identifying said sample as having said presence of said misfolded polypeptides if said fibrils are detected; and
(d3) identifying said sample as lacking said misfolded polypeptides if said fibrils are not detected; or
wherein said method comprises:
(a4) exposing said sample to metallic nanoparticles having a size of no more than 1 μm during a seeded amplification assay, wherein said seeded amplification assay comprises exposing said sample light illumination of said sample, wherein misfolded polypeptides, when present within said sample, form globular polypeptide aggregates;
(b4) detecting said globular polypeptide aggregates if formed in step (a);
(c4) identifying said sample as having said presence of said misfolded polypeptides if said globular polypeptide aggregates are detected; and
(d4) identifying said sample as lacking said misfolded polypeptides if said globular polypeptide aggregates are not detected.

90-92. (canceled)
